(19) United States
(12) Patent Application Publication
(10) Pub. No.: US 2015/0185215 A1
(43) Pub. Date: Jul. 2, 2015

(54) Cell-Based Assays For Post-Translational Enzyme Activity

(71) Applicant: Cell Assay Innovations, Inc., Beverly, MA (US)

(72) Inventors: Deborah Moshinsky, Wayland, MA (US); Liping Wu, Belmont, MA (US)

(21) Appl. No.: 14/412,454

(22) PCT Filed: Jul. 1, 2013
(86) PCT No.: PCT/US13/48887

§ 371 Date: Jan. 1, 2015

Related U.S. Application Data
(60) Provisional application No. 61/667,206, filed on Jul. 2, 2012.

(51) Int. Cl. G01N 33/573

(52) U.S. Cl. CPC G01N 33/573; G01N 2333/912; G01N 2440/14; G01N 2500/04; G01N 2500/10; G01N 2458/30

(57) ABSTRACT

Presently disclosed are cell-based methods and kits for assaying the effect of a test compound on the activity of one or more post-translational modification enzymes (e.g. kinases, methylases, etc.). The disclosed methods and kits are suitable for multiplex and/or high-throughput applications, as they are readily adaptable to assay activity of essentially any post-translational modification enzyme(s).

### REFERENCE TO RELATED APPLICATIONS

This application is based upon and claims the priority of U.S. Provisional Patent Application No. 61/667,206, entitled “Cell-Based Assay For Post-Translational Enzyme Activity,” filed Jul. 2, 2013, which is hereby incorporated in its entirety by reference.

### FIELD OF THE INVENTION

This invention relates generally to assays and methods for assessing enzyme activity in biological cells, and more specifically to assessing activity of post-translational modification enzymes.

### BACKGROUND OF THE INVENTION

The activation of proteins by post-translational modification is an important cellular mechanism for regulating most embodiments of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein post-translational modification on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 500 different protein kinases alone, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82: 111-21 (1999). A multitude of other proteins are modified by additional post-translational modifications, such as acetylation, glycosylation, and methylation.

Many of these post-translational modification sites regulate critical biological processes and may prove to be important diagnostic or therapeutic targets for molecular medicine. For example, of the more than 100 dominant oncogenes identified to date, 46 are protein kinases. See Hunter, supra. Understanding which proteins are modified, e.g. by these kinases, will greatly expand our understanding of the molecular mechanisms underlying oncogenic transformation. Therefore, the identification of, and ability to detect, post-translational modification sites on a wide variety of cellular proteins is crucially important to understanding the key signaling proteins and pathways implicated in the progression of diseases like cancer.

The development of antibodies specific for post-translational modifications (e.g. anti-phosphotyrosine antibodies, anti-acetyl-lysine antibodies) has provided a non-radioactive means for measuring enzymatic modification of particular substrate residues. For example, White and Backer (Methods in Enzymology 201: 65-67 (1991)) mention polyclonal antibodies which selectively bind to phosphotyrosine, and are considered to be useful for studying receptor tyrosine kinases (RTKs). Anti-phosphotyrosine antibodies were also used by Cleaveland et al. (Analytical Biochemistry 190: 249-253 (1990)), in their protein tyrosine kinase ELISA assay, to measure the ability of purified tyrosine kinases to phosphorylate synthetic polymeric substrates that were coated on an ELISA microtiter plate. The phosphotyrosine produced by kinase-induced phosphorylation of the polymeric substrate is then quantitated by addition of an anti-phosphotyrosine antibody, the presence of which is detected using a second rabbit anti-mouse antibody which is linked to a reporter enzyme, horseradish peroxidase.

Such post-translational modification-specific antibodies have been employed to selectively examine a diverse number of different enzyme substrates, since the population of substrates that have been post-translationally modified by an enzyme of interest can be isolated with a single modification-specific antibody. See, e.g. Rush et al., U.S. Pat. No. 7,198,896 (Apr. 3, 2007). A cell-free, quantitative kinase activity assay utilizing a two-antibody system (first and second pan antibodies recognizing different substrate epitopes) has also been described. See, e.g., Reagan et al., U.S. Pat. No. 7,888,050 (Feb. 15, 2011).

Alternative assays and methods that enable the detection of enzymatic activity in situ, in whole biological cells, are desirable for a variety of applications, for example, to allow the high-throughput assessment of drug candidates on tumor cells. Such cell-based assays are favored insofar as they reduce the possibility that altered interactions between receptors and the ligand may occur as a consequence of the receptor not being membrane-bound, and because they better mimic the response of the cells, and constituent enzymes, that can be expected in vivo. Furthermore, if the enzymes are in a multimeric complex, a cell-based assay system enables the correctly assembled enzyme to be studied. See, generally, J. Xian, Trends in Bio/Pharmaceutical Industry, (2009), available at: http://www.tbiweb.org/tbi/file_dir/TBI2009/Cell-based%20Assay.pdf.

A cell-based assay system for examining cellular serine/threonine-kinase activity utilizing a mixture of generic (promiscuous) peptide substrates and based on TR-FRET detection by localization of a rare-earth element tag has been described. See Adams et al., Curr. Chem. Genomics 1: 54-64 (2008). See also KinEASE™ system, available from CisBio International. A receptor tyrosine kinase (RTK) activation assay utilizing transformed cells and stimulation by extra-cellular ligand has been described. See Godowski et al., U.S. Pat. No. 6,287,784 (Sep. 11, 2001). Cell-based kinase assay services are also generally commercially available, for example, from ProQinase Inc. Additionally, a cell-based assay system for examining autophosphorylation of an exogenously expressed RTK has been described. See Patel et al., J. Pharmacol. Exper. Ther. 306: 838-845 (2003). These approaches are generally limited in that they are not suitable for the simultaneous, in-cell assessment of multiple post-translational modification enzymes on specific protein substrates of the particular enzymes, in a manner suitable for high throughput assessment of drug candidates, and the like. They also do not provide for a generalized methodology for assessing specific post-translational modification activity of numerous different enzymes within a target class, as the described systems are limited to a single particular target or small number of targets of interest.

To treat complex human diseases effectively, a systems-level approach is needed to understand the interplay of environmental cues, intracellular signals, and cellular behaviors that underlie disease states. This approach requires high-throughput, multiplex techniques that measure quantitative temporal variations of multiple protein activities in the intracellular signaling network. Accordingly, there remains a need for improved, simple, and robust cell-based assays and methods for assessing the activity of post-translational modification enzymes, including kinases, on their substrates in a suitable high-throughput format.

### SUMMARY OF THE INVENTION

Methods and kits for assaying the effect of a test compound on the activity of one or more enzymes are disclosed. A multiplex method for assaying the effect of a test compound on the activity of multiple kinases or one or more post-translational modification enzymes in a biological cell is disclosed. The method can include (a) obtaining a plurality of transiently transfected biological cells that transiently express (i) at least a first target kinase and a second target kinase, and (ii) at least one first specific protein substrate of the first target kinase and at least one second specific protein substrate of the second target kinase, wherein the first specific protein substrate is linked to at least one first capture tag and the second specific protein substrate is linked to at least one second capture tag, and wherein the first and second capture tags are not conjugated to rare earth elements and are not identical; (b) culturing the plurality of biological cells under conditions suitable to transiently express the first and second target kinases and the first and second specific protein substrates; (c) incubating the plurality of biological cells, in at least one first multi-well plate, with at least one test compound under conditions suitable to allow phosphorylation of the first and second specific protein substrates by the first and second target kinases; (d) lysing the plurality of biological cells, transferring the lysed cells to at least one second multi-well plate, and capturing (i) the first specific protein substrate using at least one capture antibody that specifically binds the first capture tag, and (ii) the second specific protein substrate using at least one capture antibody that specifically binds the second capture tag; (e) detecting the presence of phosphorylated first and second specific protein substrates using (i) at least one first detectable antibody that specifically binds the phosphorylated form of the first specific protein substrate, but does not substantially bind the first specific protein substrate when not phosphorylated, and (ii) at least one second detectable antibody that specifically binds the phosphorylated form of the second specific protein substrate, but does not substantially bind the second specific protein substrate when not phosphorylated; and (f) comparing the level of phosphorylated first and second specific protein substrates detected in step (e) with 1) the level of phosphorylated first and second specific protein substrates in at least one control sample taken from control biological cells not contacted with the test compound, and 2) the level of phosphorylated first and second specific protein substrates in at least one control sample taken from control biological cells not contacted with the test compound but rather treated with known target kinase inhibitor(s), or otherwise equivalent cells expressing kinase-deficient mutant instead of active forms of the target kinases, thereby assaying the effect of the test compound on the activity of the first and second target kinases within the biological cells of step (c).

In another embodiment, the method can include (a) obtaining a plurality of transiently transfected biological cells that transiently express (i) at least one target post-translational modification enzyme and (ii) at least one specific protein substrate of the target post-translational modification enzyme, wherein the specific protein substrate is linked to at least one capture tag that is not conjugated to a rare earth element; (b) culturing the plurality of biological cells under conditions suitable to transiently express the target post-translational modification enzyme and the specific protein substrate; (c) incubating the plurality of biological cells with at least one test compound under conditions suitable to allow post-translational modification of the specific protein substrate by the target post-translational modification enzyme; (d) lysing the plurality of biological cells and capturing the specific protein substrate using at least one capture antibody that specifically binds the capture tag; (e) detecting the presence of post-translationally modified specific protein substrate using at least one detection antibody that specifically binds the post-translationally-modified form of the specific protein substrate but does not substantially bind the specific protein substrate when not post-translationally modified; and (f) comparing the level of post-translationally modified specific protein substrate detected in step (e) with 1) the level of post-translationally modified specific protein substrate in at least one control sample taken from control biological cells not contacted with the test compound, and 2) the level of post-translationally modified specific protein substrate in at least one control sample taken from control biological cells not contacted with the test compound but rather treated with known target post-translational modification enzyme inhibitor(s), or otherwise equivalent cells expressing enzyme activity-deficient mutant instead of active forms of the target post-translational modification enzyme(s), thereby assaying the effect of the test compound on the activity of the target post-translational modification enzyme within the biological cells of step (c).

Also included are target enzymes. The target enzyme can include multiple target kinases, post-translational modification enzymes, serine/threonine kinases, mutant kinases and drug-resistant kinases. The multiple target kinases can be tyrosine kinases or serine/threonine kinases. The tyrosine kinase can be a receptor tyrosine kinase selected from an ALK, AXL, DDR, EGFR, EPH, FGFR, INSR, MET, MUSK, PDGFR, PTK7, RET, ROR, ROS, RYK, TIE, TRK, VEGFR, and AATYK family kinase. The tyrosine kinase can be a cytoplasmic tyrosine kinase selected from an ABL, ACK, CSK, FAK, FES, FRK, JAK, SRC, TEC, and SYK family kinase. The tyrosine kinase or serine/threonine kinase can be a tyrosine kinase like (TKL) kinase selected from an MLK, RAF, STKR, LRRK, LISK, IRAK, and RIPK family kinase. The serine/threonine kinase can be an AGC family kinase selected from a AktR, Akt, GRK, MAST, DMPK, NDR, PDK1, PKA, PKC, PKG, PKN, RSK, RSKL, RSKR, SGK, AGC-Unique, YANK, AGC-Sar, AGC1, AGC, and PTF kinase. The serine/threonine kinase can be a CAMK family kinase selected from a CDPK, CAMKL, CAMK1, CAMK2, DAPK, DCAMKL, MAPKAPK, MLCK, Trb1, PHK, PKD, PSK, RSKb, CAMK-Unique, CAMK-Tt, CASK, PIM, RAD53, Trio, TSSK, Ciliate-C1, STK33, SgK495, CAMKX, CAMKY, Chk1L, MLCK-like, PPCK, CAMK-Tvag1, CAMK-Tvag2, and CAMK-Tvag3 kinase. The serine/threonine kinase can be a CK1 family kinase selected from a CK1, Dual, TTBK, Worm10, Worm11, Worm7, Worm8, Worm9, TTBKL, VRK, and CK1-Unique kinase. The serine/threonine kinase can be a CMGC family kinase selected from a Dicty1, CLK, CK2, CDK, CDKL, DYRK, GSK, MAPK, SRPK, CMGC-Unique, RCK, CMGC-GL1, and CMGC-Tvag1 kinase. The serine/threonine kinase can be a STE family kinase selected from a Dicty2, Dicty3, STET, STE11, STE20, Ste-Unique, and STE-plant1 kinase. The serine/threonine kinase can be an Atypical family kinase selected from a AFK, A6, BCR, BRD, HisK, PDHK, TIF1, FAST, G11, TAF1, H11, and PI3 kinase. The serine/threonine kinase can be a Worm6, Dicty10, Dicty6, Dicty7, Dicty8, Dicty9, Aur, CAMKK, CDC7, urch, WEE, IKK, IRE, IKS, NEK, Slob, NKF1, NKF2, NKF3, NKF4, NKF5, NRBP, NAK, PLK, SAMK, TBCK, Worm1, Worm2, Worm3, Worm4, Worm5, Other-Unique, TLK, Ciliate-A1, Ciliate-A2, Ciliate-A3, Ciliate-A4, Ciliate-A5, Ciliate-A6, Ciliate-A7, Ciliate-A8, Ciliate-A9, Ciliate-B1, Ciliate-B2, Ciliate-B3, Ciliate-C8, Ciliate-C3, Ciliate-C4, Ciliate-C5, Ciliate-C6, Ciliate-C7, Ciliate-D1, Ciliate-E1, Ciliate-E2, Ciliate-E3, Ciliate-E4, Ciliate-E5, Ciliate-E6, Ciliate-E7, Ciliate-E8, Ciliate-E9, Ciliate-F1, Ciliate-F2, WNK, Bub, Bud32, HAL, Haspin, MOS, PEK, RAN, SCY1, TOPK, TTK, ULK, VPS15, Ciliate-G, Ciliate-Unique, Ciliate_C8_Cterm, Dusty, KIS, SgK071, SgK493, SgK496, Unil, AgaK1, FunK1, HisK, Mak2, Mbre3, NEK-frag, NEK-like, Other-GL1, Other-GL2, Other-Tvag1, Other-Tvag3, and Other-Tvag2 kinase. In one embodiment, the serine/threonine kinase is a receptor guanylate cyclase (RGC) family kinase. The post-translational modification enzyme can be a methylase, acetylase, glycosylase and the post-translational modification is methylation, acetylation and glycosylation, respectively. In another embodiment, the post-translational modification enzyme is an enzyme selected from Column 1, Table 1, and the corresponding post-translational modification is selected from the corresponding row of Column 2, Table 1.

Two or more different target enzymes can include at least one tyrosine kinase and at least one serine/threonine kinase. Two or more different target post-translational modification enzymes can include tyrosine kinases, serine/threonine kinases, and combinations thereof. The target kinases can auto-phosphorylate a domain of any of the kinases, phosphorylate specific peptide substrates, phosphorylate an independent non-domain substrate, or a combination thereof. The post-translational modification enzymes may also be kinases, such as a tyrosine kinase or serine/threonine kinase, where the post-translational modification is phosphorylation. Activities of the kinases may also be assayed, such as through non-endogenous kinase activities. Activities of the post-translational modification enzymes can be assayed through substantially non-endogenous post-translational modification enzyme activities.

Specific protein substrates are also disclosed. In one embodiment, specific protein substrate(s) may not be a plurality of promiscuous peptide substrates capable of being phosphorylated by the target kinases or other kinases or post-translationally modified by a plurality of post-translational modification enzymes. In another embodiment, the individual specific protein substrates can be a domain of the tyrosine kinase that is auto-phosphorylated by the kinase domain of the tyrosine kinase. The specific protein substrate can be (i) a domain of the serine/threonine kinase that is auto-phosphorylated by the kinase domain of the serine/threonine kinase or (ii) not a domain of the serine/threonine kinase. The specific protein substrate can be BAD.

The biological cells can be human primary cells, human stem cells, human cell lines, or combinations thereof. In particular, the biological cells can be human embryonic kidney (HEK) 293 cells. The biological cells can be previously transiently transfected with at least one polynucleotide including a polynucleotide encoding the tyrosine kinase. The biological cells can be co-transfected with at least one first polynucleotide including a polynucleotide encoding the serine/threonine kinase or tyrosine kinase and at least one second polynucleotide including a polynucleotide encoding the specific protein substrate, at least one second polynucleotide including a polynucleotide encoding the first specific protein substrate, at least one third polynucleotide including a polynucleotide encoding the second serine/threonine kinase or tyrosine kinase, or at least one fourth polynucleotide including a polynucleotide encoding the second specific protein substrate. The biological cells can also be serum starved prior to transfection. The biological cells can include an expression vector with one or more polynucleotides encoding one or more target kinases. The biological cells can be capable of expressing the target enzyme and the specific protein substrate within about 1-5 days of being transfected, and the transiently transfected biological cells are not stably transformed and do not stably express the target enzyme or the specific protein substrate, other than in endogenous form.

Polynucleotides can be also included. A first and second polynucleotide can be operably linked to express one or more fusion proteins including a kinase and the specific protein substrate. A third and fourth polynucleotide may be included that are operably linked to express additional fusion proteins including an additional kinase, such as a serine/threonine or tyrosine kinase, and an additional specific protein substrate. The polynucleotides can also include a single expression vector.

Capture tags are also included. The capture tag can be selected from the FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, Glu-Glu, or combinations thereof.

One or more detection antibodies are also included. The detection antibodies can detect the same or different specific protein substrate. The detection antibody can include a detectable label. The detectable label can be horse radish peroxidase, quantum dots, fluorophores, alkaline phosphatase, phospho-tyrosine specific antibody and combinations thereof. The detection antibody can be a primary antibody that is specifically bound by a secondary antibody, where the secondary antibody includes a detectable label or is bound to another molecule that includes one or more detectable labels. The detection antibody can be a phosphorylation-site specific antibody that specifically binds the specific protein substrate when phosphorylated at a particular serine or threonine or tyrosine site but does not substantially bind the specific protein substrate when not phosphorylated at that particular serine or threonine or tyrosine site. In one embodiment, the detection antibody can be a phosphorylation-site specific antibody such as phospho-BAD (serine 112) antibody. In another embodiment, the detection antibody is a phospho-tyrosine specific antibody. Multiple detection antibodies can be included. A first and second primary antibody can bind to (i) first and second secondary antibody, where each includes at least one detectable label or (ii) binds to a first and second molecule each including at least one detectable label.

The method can be carried out in a multi-well plate. The method can further include transferring the lysed biological cells to a multi-well plate, and the capture antibody can be affixed to a plurality of wells in the plate or to a plurality of beads added to the plate. The method can also be employed in multiplex fashion utilizing two or more different, target post-translational modification enzymes and two or more different, specific protein substrates to assay the effect of at least one test compound on the activity of the two or more different, target post-translational modification enzymes.

Also included are kits for assaying the effect of a test compound on the activity one or more enzymes, such as kinases or more post-translational modification enzymes, in a biological cell. In one embodiment, the kit can include (a) a plurality of human embryonic kidney (HEK) 293 cells transiently transfected with one or more expression vectors comprising a plurality of polynucleotides encoding (i) a plurality of target kinases and (ii) a plurality of specific protein substrates of the target kinases, wherein each specific protein substrate is linked to at least one unique capture tag that is not conjugated to a rare earth element; (b) one or more multi-well capture plates or beads comprising a plurality of capture antibodies, each of which specifically binds to one of the unique capture tags linked to a specific protein substrate; (c) optionally, a plurality of human embryonic kidney (HEK) 293 cells transiently transfected with one or more expression vectors comprising a plurality of polynucleotides encoding (i) a plurality of target inactive mutant enzymes and (ii) a plurality of specific protein substrates of the target kinases, wherein each specific protein substrate is linked to at least one unique capture tag that is not conjugated to a rare earth element, and wherein the expression vectors are suitable for transiently transfecting the cells; (d) optionally, one or more reagents for culturing and/or lysing the cells transiently transfected with the expression vectors; (e) optionally, a plurality of labeled detection antibodies each of which specifically binds to the phosphorylated form of a specific protein substrate but does not substantially bind to the un-phosphorylated form of that specific protein substrate; (f) optionally, one or more control compounds for inhibiting one or more of the target kinases; and (g) instructions for (i) culturing the transfected cells under conditions suitable to transiently express the plurality of target kinases and the plurality of specific protein substrates, (ii) plating the cells in multi-well plates, contacting the cells with at least one test compound, and incubating the cells under conditions suitable to allow phosphorylation of the specific protein substrates by the target kinases, (iii) lysing the cells, transferring the lysed cells to the multi-well capture plate of step (b), and capturing the plurality of specific protein substrates on or in the multi-well capture plate, (iv) detecting the presence of phosphorylated forms of the plurality of specific protein substrates using the plurality of labeled detection antibodies; and (v) comparing the level of phosphorylated forms of the plurality of specific protein substrates detected in a test sample of cells contacted with the test compound with 1) the level of phosphorylated forms of the plurality of specific protein substrates in at least one control sample taken from control HEK 293 cells not contacted with the test compound, and 2) the level of phosphorylated forms of the plurality of specific protein substrates in at least one control sample taken from control HEK 293 cells not contacted with the test compound but rather treated with known target kinase inhibitor(s), or otherwise equivalent cells expressing kinase-deficient mutant instead of active forms of the target kinases, thereby assaying the effect of the test compound on the activity of the plurality of target kinases within the cells contacted with the test compound. Another embodiment includes a kit for assaying the effect of a test compound on the activity of one or more post-translational modification enzymes in a biological cell, the kit comprising: (a) a plurality of human embryonic kidney (HEK) 293 cells transiently transfected with one or more expression vectors comprising a plurality of polynucleotides encoding (i) a plurality of target post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells; (b) one or more multi-well capture plates or beads comprising a plurality of capture antibodies, each of which specifically binds to one of the unique capture tags linked to a specific protein substrate; (c) optionally, a plurality of human embryonic kidney (HEK) 293 cells transiently transfected with one or more expression vectors comprising a plurality of polynucleotides encoding (i) a plurality of target inactive mutant post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells; (d) optionally, one or more reagents for culturing and/or lysing the cells transiently transfected with the expression vectors; (e) optionally, a plurality of labeled detection antibodies each of which specifically binds to the post-translationally modified form of a specific protein substrate but does not substantially bind to the unmodified form of that specific protein substrate; (f) optionally, one or more control compounds for inhibiting the target post-translational modification enzymes; and (g) instructions for (i) culturing the transfected cells under conditions suitable to transiently express the plurality of target post-translational modification enzymes and the plurality of specific protein substrates, (ii) plating the cells in multi-well plates, contacting the cells with at least one test compound, and incubating the cells under conditions suitable to allow post-translational modification of the specific protein substrates by the target post-translational modification enzymes, (iii) lysing the cells, transferring the lysed cells to the multi-well capture plate, and capturing the plurality of specific protein substrates on or in the multi-well capture plate, (iv) detecting the presence of post-translationally modified forms of the plurality of specific protein substrates using the plurality of labeled detection antibodies; and (v) comparing the level of post-translationally modified forms of the plurality of specific protein substrates detected in a test sample of cells contacted with the test compound with 1) the level of post-translationally modified forms of the plurality of specific protein substrates in at least one control sample taken from control HEK 293 cells not contacted with the test compound, and 2) the level of post-translationally modified forms of the plurality of specific protein substrates in at least one control sample taken from control HEK 293 cells not contacted with the test compound but rather treated with known target post-translational modification enzyme inhibitor(s), or otherwise equivalent cells expressing enzyme activity-deficient mutant instead of active forms of the target post-translational modification enzymes, thereby assaying the effect of the test compound on the activity of the plurality of post-translational modification enzymes within the cells contacted with the test compound.

The kit can further include one or more capture antibodies. The capture antibodies can be bound to a multi-well capture plate or conjugated to beads that are not bound to the multi-well capture plate.

### BRIEF DESCRIPTION OF THE DRAWINGS

FIG. 1 is a graphic representation of the generalized, cell-based assay strategy disclosed herein;

FIG. 2 shows expression of lck-FLAG and K273M lck-FLAG in transiently transfected 293F cells by Western Blot;

FIG. 3 shows phosphorylation of lck-FLAG over background of K273M lck-FLAG in transiently transfected 293F cells by Western Blot;

FIG. 4 shows dose response curves for dasatinib, a known lck inhibitor, and U0126, a MEK inhibitor that should not exhibit activity against lck;

FIG. 5 shows expression of Pim-2 and K61M Pim-2 in transiently transfected 293F cells by Western Blot;

FIG. 6 shows expression of BAD-FLAG in transiently transfected 293F cells by Western Blot;

FIG. 7 shows expression of BAD-FLAG in transiently transfected 293F cells after anti-FLAG immunoprecipitation by Western Blot;

FIG. 8 shows phosphorylation of BAD-FLAG in transiently transfected 293F cells by Western Blot;

FIG. 9A shows dose response curve against Pim-2/BAD for staurosporine, a general kinase inhibitor; and

FIG. 9B shows the dose response curve against Pim-2/BAD for SGI-1776, a more specific inhibitor that should exhibit activity against Pim-2.

### DETAILED DESCRIPTION OF THE INVENTION

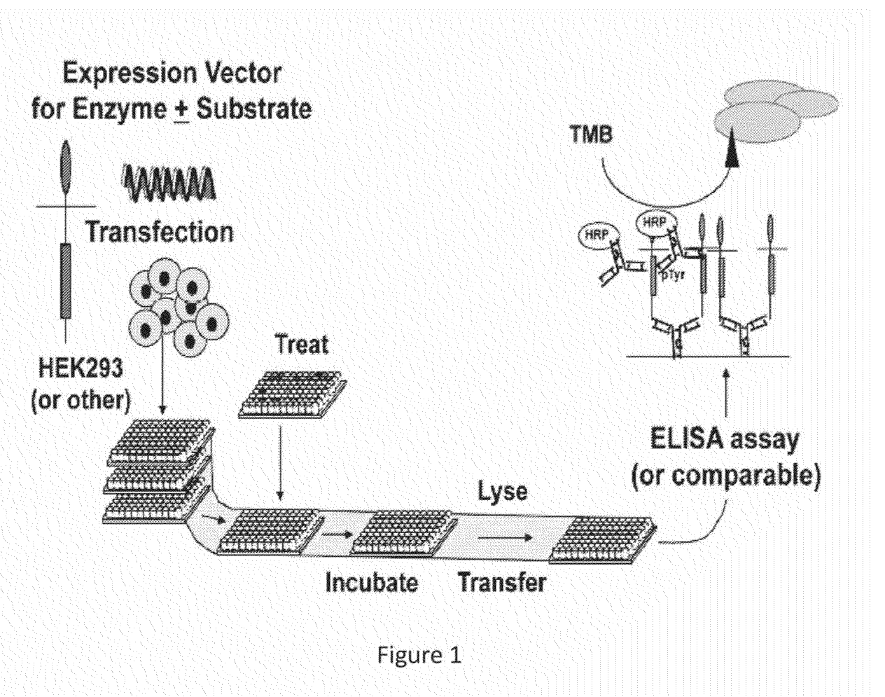

The invention provides cell-based methods and kits for assaying the effect of a test compound on the activity of one or more post-translational modification enzymes. See FIG. 1. The disclosed methods and kits are suitable for multiplex and/or high-throughput applications, as they are readily adaptable to assay activity of essentially any post-translational modification enzyme(s), as further described and disclosed herein.

Certain exemplary embodiments will be described to provide an overall understanding of the principles of the structure, function, manufacture, and use of the devices and methods disclosed herein. Those skilled in the art will understand that the devices and methods specifically described herein are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In one embodiment, the invention provides a multiplex method for assaying the effect of a test compound on the activity of multiple kinases in a biological cell, the method comprising the steps of:

(a) obtaining a plurality of transiently transfected biological cells that transiently express (i) at least a first target kinase and a second target kinase, and (ii) at least one first specific protein substrate of the first target kinase and at least one second specific protein substrate of the second target kinase, wherein the first specific protein substrate is linked to at least one first capture tag and the second specific protein substrate is linked to at least one second capture tag, and wherein the first and second capture tags are not conjugated to rare earth elements and are not identical;

(b) culturing the plurality of biological cells under conditions suitable to transiently express the first and second target kinases and the first and second specific protein substrates;

(c) incubating the plurality of biological cells, in at least one first multi-well plate, with at least one test compound under conditions suitable to allow phosphorylation of the first and second specific protein substrates by the first and second target kinases;

(d) lysing the plurality of biological cells, transferring the lysed cells to at least one second multi-well plate, and capturing (i) the first specific protein substrate using at least one capture antibody that specifically binds the first capture tag, and (ii) the second specific protein substrate using at least one capture antibody that specifically binds the second capture tag;

(e) detecting the presence of phosphorylated first and second specific protein substrates using (i) at least one first detectable antibody that specifically binds the phosphorylated form of the first specific protein substrate, but does not substantially bind the first specific protein substrate when not phosphorylated, and (ii) at least one second detectable antibody that specifically binds the phosphorylated form of the second specific protein substrate, but does not substantially bind the second specific protein substrate when not phosphorylated; and

(f) comparing the level of phosphorylated first and second specific protein substrates detected in step (e) with 1) the level of phosphorylated first and second specific protein substrates in at least one control sample taken from control biological cells not contacted with the test compound, and 2) the level of phosphorylated first and second specific protein substrates in at least one control sample taken from control biological cells not contacted with the test compound but rather treated with known target kinase inhibitor(s), or otherwise equivalent cells expressing kinase-deficient mutant instead of active forms of the target kinases, thereby assaying the effect of the test compound on the activity of the first and second target kinases within the biological cells of step (c).

In an embodiment of the multiplex method, the first and second specific protein substrates are each not a plurality of promiscuous peptide substrates capable of being phosphorylated by the first and second target kinases or other kinases. In another embodiment, the first and second target kinases are tyrosine kinases, wherein the first specific protein substrate is a domain of the first tyrosine kinase that is auto-phosphorylated by the kinase domain of the first tyrosine kinase, and wherein the second specific protein substrate is a domain of the second tyrosine kinase that is auto-phosphorylated by the kinase domain of the second tyrosine kinase.

In another embodiment of the multiplex method, the first and second target kinases are serine/threonine kinases, wherein the first specific protein substrate is (i) a domain of the first serine/threonine kinase that is auto-phosphorylated by the first serine/threonine kinase or (ii) an independent non-domain substrate that is phosphorylated by the first serine/threonine kinase, wherein the second specific protein substrate is (iii) a domain of the second serine/threonine kinase that is auto-phosphorylated by the first serine/threonine kinase or (iv) an independent non-domain substrate that is phosphorylated by the second serine/threonine kinase, and wherein the first and second specific protein substrates are not identical with respect to either sequence, phosphorylation site, epitope tag, or any combination of the 3.

In yet another embodiment of the multiplex method, the biological cells of step (a) have been transiently co-transfected with (i) at least one first polynucleotide comprising a polynucleotide encoding the first serine/threonine kinase, (ii) at least one second polynucleotide comprising a polynucleotide encoding the first specific protein substrate, (iii) at least one third polynucleotide comprising a polynucleotide encoding the second serine/threonine kinase, and optionally (iv) at least one fourth polynucleotide comprising a polynucleotide encoding the second specific protein substrate.

In another embodiment of the multiplex method, the first and second polynucleotides are operably linked to express a first fusion protein comprising the first serine/threonine kinase and the first specific protein substrate, and wherein the third and fourth polynucleotides are operably linked to express a second fusion protein comprising the second serine/threonine kinase and the second specific protein substrate. In still another embodiment, the first target kinase is a tyrosine kinase, wherein the second target kinase is a serine/threonine kinase, wherein the first specific protein substrate is a domain of the tyrosine kinase that is auto-phosphorylated by the kinase domain of the tyrosine kinase, and wherein the second specific protein substrate is (i) a domain of the serine/threonine kinase that is auto-phosphorylated by serine/threonine kinase or (ii) an independent non-domain substrate that is phosphorylated by the serine/threonine kinase.

In a further embodiment of the multiplex method, the biological cells of step (a) have been transiently co-transfected with (i) at least one first polynucleotide comprising a polynucleotide encoding the tyrosine kinase, (ii) at least one second polynucleotide comprising a polynucleotide encoding the serine/threonine kinase, and optionally (iii) at least one third polynucleotide comprising a polynucleotide encoding the specific protein substrate of the serine/threonine kinase. In another embodiment, the second and third polynucleotides are operably linked to express a fusion protein comprising the serine/threonine kinase and the specific protein substrate. In some embodiments of the multiplex method, the biological cells of step (a) have been serum starved prior to being transiently co-transfected.

In still another embodiment of the multiplex method, the transiently transfected biological cells comprise an expression vector that comprises first polynucleotide encoding the first target kinase and a second polynucleotide encoding the second target kinase. In another embodiment, the first, second, third, and fourth polynucleotides are comprised within a single expression vector. In yet another embodiment, the first, second, and third polynucleotides are comprised within a single expression vector.

In yet another embodiment of the multiplex method, the transiently transfected biological cells of step (a) comprise human embryonic kidney (HEK) 293 cells capable of expressing the first and second target kinases, and the first and second specific substrates, within about 1-5 days of being transfected, and wherein the transiently transfected HEK 293 cells are not stably transformed and do not stably express the first and second target kinases or the first and second specific protein substrates, except in endogenous form.

In another embodiment of the multiplex method, the first and second capture tags are selected from the group consisting of FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, and Glu-Glu, or combinations thereof

Generally, a suitable capture tag comprises a short polypeptide which has enough residues to provide an epitope (preferably a linear epitope) against which a capture antibody can specifically bind and recognize the tag, yet is short enough such that it does not interfere with activity of the protein to which it is affixed. The capture tag is also sufficiently unique so that the capture antibody specific for the tag does not bind to other reagents (including un-tagged proteins) in the assay. Selection of a “unique” capture tag sequence can be accomplished by comparing the sequence of a proposed tag polypeptide against other known sequences in Genbank or EMBL, for example. Suitable capture tags generally have at least 6 amino acid residues and usually between about 8-80 amino acid residues (preferably between about 9-30 amino acid residues).

The capture tag is provided at a location in the receptor construct such that: a) the tag does not interfere with ligand or substrate binding to the target enzyme b) the tag does not interfere with autophosphorylation activities of a target kinase; c) the tag is presented in a suitable configuration so that it can bind to the capture antibody in ELISA or bead-based assay formats. Often, the capture tag will be present at the N-terminus of the substrate construct. Alternatively, the capture tag may be present at the C-terminus of the receptor construct.

In a further embodiment of the multiplex method, the first and second detection antibodies of step (e) each comprise a detectable label selected from the group consisting of horse radish peroxidase, quantum dots, fluorophores, alkaline phosphatase, or combinations thereof. In yet a further embodiment, the first and second detection antibodies of step (e) are first and second primary antibodies, wherein the first and second primary antibodies bind to (i) first and second secondary antibodies each comprising at least one detectable label or (ii) bind to first and second molecules each comprising at least one detectable label. And, in yet another embodiment, the first and second detection antibodies of step (e) are phospho-tyrosine specific antibodies, and may be the same antibody.

In another embodiment of the multiplex method, (i) the first detection antibody of step (e) is a phosphorylation-site specific antibody that specifically binds the first specific protein substrate when phosphorylated at a particular serine or threonine site but does not substantially bind the first specific protein substrate when not phosphorylated at that particular serine or threonine site, and wherein (ii) the second detection antibody of step (e) is a phosphorylation-site specific antibody that specifically binds the second specific protein substrate when phosphorylated at a particular serine or threonine site but does not substantially bind the second specific protein substrate when not phosphorylated at that particular serine or threonine site.

In yet another embodiment of the multiplex method, the first detection antibody of step (e) is a phospho-tyrosine specific antibody, and wherein the second detection antibody of step (e) is a phosphorylation-site specific antibody that specifically binds the second specific protein substrate when phosphorylated at a particular serine or threonine site but does not substantially bind the second specific protein substrate when not phosphorylated at that particular serine or threonine site. In yet another embodiment, the activities of the first and second target kinases that are assayed are substantially non-endogenous kinase activities. While in still another embodiment, either or both of the first and second target kinases are mutant kinases or drug-resistant kinases.

Multiplex immunological assays are well described and known to those of skill in the art. The ability to detect and assess compound (e.g. drug candidate) effect on a number of different of proteins is a critical step in proteomics and the drug discovery process. Multiplexed analysis offers significant advantages regarding time, reagent cost, sample requirements and the amount of data that can be generated. Typically, an antibody-based multiplex assay may suitably detect as few as 2 and as many as 5 or more different target analytes (e.g. enzymes or their activity on substrates). See generally, e.g., Fu et al., Proteomics 4(3): 271-284 (2010); see also Janes et al., Molec. and Cell. Proteomics 2: 463-473 (2003), and Yu et al., PNAS 106(28): 11606-11611 (2009). Luminogenic multiplex assays for enzyme activity are also known in the art. See, e.g. Riss et al., U.S. Pat. No. 7,416,854 (2008). Similarly, multiplex kinase activity assays employing peptide substrates have been described. See, e.g., Shults et al., ChemBioChem 8: 933-942 (2007); see also Adams et al., Curr. Chem. Genomics (2008), supra.

The impact of test compounds on the activity of post-translational modification enzymes (i.e. their ability to modify (e.g. phosphorylate) their protein substrate(s)) may be assayed for a variety of different drugs and enzymes. For example, drug candidates may be antagonists or agonists of their target enzyme, either inhibiting or enhancing post-translational modification activity. The compound may be a specific enzyme inhibitor, such as an AKT kinase targeted inhibitor, or a “pan” kinase inhibitor that inhibits more than type of kinase (for example, bis-indoleimide). Inhibitory compounds of interest may be targeted inhibitors that modulate post-translational activity of the target enzyme, or may be upstream expression inhibitors, such as siRNA or anti-sense inhibitors. Such compound may indirectly inhibit target enzyme activity by, e.g., inhibiting another kinase that phosphorylates and thus activates the target kinase of interest, or by inhibiting co-factors or necessary binding partners or complex partners of the target enzyme (e.g. a target kinase).

The methods of the invention may be carried out in any number of well-known assay formats suitable for multi-well plate format. Immunoassay formats and variations thereof, which may be useful for carrying out the methods disclosed herein, are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al, “Immunometric Assays Using Monoclonal Antibodies”). Conditions suitable for the formation of reagent-antibody complexes are well described. See id. A review of ELISA is found in Current Protocols in Molecular Biology, Vol. 2, chapter 11 (1991).

Solid-phase ELISA and bead-based detection assays are desirable for the practice of the presently disclosed methods. Capture antibodies specific for an employed capture tag on a desired protein substrate (e.g. a substrate of a target serine/threonine kinase) may be bound to multi-well plates (e.g. micotiter plates) or bound to beads (e.g. xMAP system from Luminex Corporation). In the plate assay format, the capture antibody is immobilized on the capture plate and the captured protein substrate remains immobilized on the capture plate for subsequent detection. In the bead assay format, the capture antibody (bound to the bead) is free in solution contained in the capture plate, and the captured protein substrate remains immobilized on the bead for subsequent detection.

Detection antibodies specific for the post-translationally modified form of the desired protein substrate may comprise a detectable label (such as quantum dots, alkaline phosphatase, fluorphores, etc.) or may be a primary antibody that is bound by a secondary detectable antibody, itself comprising a detectable label.

Multi-well plates useful in the practice of the disclosed methods and kits may be “microtiter” plates. The term “microtiter” plate when used herein refers to an assay plate having between about 30 to 1600 individual wells, often 96 wells. Often, the individual wells of the microtiter plate will hold a maximum volume of about 250 uL. Conveniently, the first assay plate is a 96 well polystyrene or plastic, cell culture microtiter plate (such as that sold by Becton Dickinson Labware, Lincoln Park, N.J.), which allows for automation. Often, about 50 uL to 300 uL, more preferably 100 uL to 200 uL, of an aqueous sample comprising cell culture media with the cells suspended therein will be added to each well of the first assay plate in the first stage of the assay (exposure of plated cells to test compound(s)). It is desirable to seed between about 1×10⁴ to 3×10⁵ cells per well. More preferably, 5×10⁴ to 1×10⁵ cells per well are seeded. Usually, the second assay plate (for capture) will comprise a polystyrene microtiter ELISA plate such as that sold by Nunc Maxisorp, Inter Med, Denmark.

Bead-based, multiplex assay methodologies are well known in the field and have been described. See, e.g., Sylvester et al., Molec. Canc. Ther. 9: 1469 (2010); Zhou et al., Anal. Biochem. 408(1): 5-11 (2011) (employing a magnetic bead system). Several bead-based systems and their components are commercially available for purchase from a number of well-known vendors, including Luminex Corporation (xMAP bead system), Perkin Elmer (AlphaScreen SureFire system), and Qiagen (LiquiChip system).

Post-translational modification enzymes and the modifications they introduce to their substrates are well known in the art, and any number of these enzymes may be employed in the methods of the present invention. For example, the enzyme may be a tyrosine kinase or a serine/threonine kinase, and the post-translational modification may be phosphorylation. Kinases and their substrates within cellular signaling pathways are well described. Alternatively, the enzyme may be an acetyltransferase and the modification may be acetylation; or the enzyme may be a glycosylase and the modification may be glycosylation; or the enzyme may be a methylase, and the modification may be methylation. Additional exemplary post-translational modification enzymes (and their corresponding substrate modifications) useful in the practice of the disclosed methods are shown in Table 1 below.

TABLE 1
Post-translational modification enzymes

| Enzyme | Modification |
|---|---|
| Palmitoyl protein thioesterases | Palmitoylation |
| Farnesyl transferase, Caax protease | Prenylation |
| and methyl transferase | |
| N-myristoyltransferase (NMT) | Myristoylation |
| Acetyltransferases | Propionylation |
| Acetyltransferases | Butyrylation |
| Unknown | Malonylation |
| Unknown | Succinylation |
| Hydroxylases | Hydroxylation |
| Sulfotransferase | Sulfation |
| Amidating enzymes | Amidation |
| Biotin ligase | Biotinylation |
| E1, E2, E3, or Ubiquitin ligase | Ubiquitination |
| | (ubiquitylation) |
| E1, E2, E3 | SUMOylation |

Preferred kinases whose activities may be assayed by the methods of the present disclosure are further described below. In some embodiments, the enzyme being assayed is a mutant kinase or a drug-resistant kinase. This is particularly useful in the cancer field, where a number of kinase mutations (including translocation mutants) have been identified as either increasing or decreasing the effectiveness of the drug against the cancer cells or the resistance/responsiveness of the tumor to the drug. The disclosed methods and kits are, therefore, useful in screening for new compounds that have efficacy against such drug-resistant mutant enzymes and cancer cells.

Examples of protein tyrosine phosphatases that may be assayed by the present methods include PTP1B, PTPMEG, PTP1c, Yop51, VH1, cdc25, CD45, HLAR, PTP18, HPTP-alpha, and DPTP10D. See Zhang and Dixon, Adv. Enzym. 68: 1-36 (1994). Examples of protein serine-threonine phosphatases include PP1, PP2A, PP2B and PP2C. See Methods in Enzymology 201: 389-398 (Hunter & Sefton eds.), Academic Press, New York, (1991).

Any mammalian cells of interest may be advantageously employed in the methods of the invention, depending on the tissue or cell type for which it is desired to test the effect on compounds on the post-translational modification enzyme activity within those cells. For example, human primary cells, human stem cells, human cells lines, or combinations thereof may be employed. Cancer cells or cell lines may, for example, be selected for examining the effect of, e.g. targeted kinase inhibitors on cellular signaling enzymes (including kinases) within those cells. One preferred cell type is human embryonic kidney (HEK) 293 cells. The chosen cells should be capable of being transiently transfected in accordance with the disclosed methods. Suitable cells are capable of expressing transfected genes within 1-5 days of being transfected, but do not stably or permanently express the transfected genes. In some embodiments of the disclosed methods, the cells may be serum starved prior to being transfected. Where more than one kinase is being transfected, the method may employ transfection of two cell populations, each transfected with a single kinase and, if required, its specific protein substrate, followed by mixing the cell populations prior to contact with test compound(s).

There are various methods of introducing foreign DNA into a eukaryotic cell for transient transfection: some rely on physical treatment (electroporation, nanoparticles, magnetofection), other on chemical materials or biological particles (viruses) that are used as carriers. These techniques are well described in the art. See, e.g. Chalberg et al., Transfection of DNA into Mammalian Cells in Culture, eLS (2005)(DOI: 10.1038/npg.els.0003928); see also Davis et al., BASIC METHODS IN MOLECULAR BIOLOGY (1986). Briefly, these approaches include chemical-based transfection (calcium phosphate, cyclodextrin, polymers, liposomes, or nanoparticles (with or without chemical or viral functionalization), non-chemical transfection (electroporation, optical transfection, gene electrotransfer, impalefecation, or hydrodynamic injection), or particle-based methods (gene gun, magnet assisted transfection, or nanofiber/nanowire impalefecation). Transfection kits and reagents are commercially available for purchase from a number of vendors, including Life Technologies, Mirus Bio, and Clonetech.

Polynucleotides encoding target enzyme(s) and/or their specific protein substrates may be contained within a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. Preferred are vectors comprising cis-acting control regions to the polynucleotide of interest. Appropriate trans-acting factors may be supplied by the host, supplied by a complementing vector or supplied by the vector itself upon introduction into the host. In certain preferred embodiments in this regard, the vectors provide for specific expression, which may be inducible and/or cell type-specific. Particularly preferred among such vectors are those inducible by environmental factors that are easy to manipulate, such as temperature and nutrient additives. Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert comprising an polynucleotide encoding a target post-translational modification enzyme and/or its specific protein substrate should be operatively linked to an appropriate promoter, such as the phage lambda PL promoter, the E. coli lac, tip and tac promoters, the SV40 early and late promoters and promoters of retroviral LTRs, to name a few. Other suitable promoters are known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated. The expression vectors may include at least one selectable marker. Such markers include dihydrofolate reductase or neomycin resistance for eukaryotic cell culture. Appropriate culture media and conditions for the above-described host cells are known in the art. See, e.g., CURRENT PROTOCOLS IN MOLECULAR BIOLOGY, Ausubel et al., eds., Volume 2, Chapter 16, Wiley Interscience.

Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter. Such materials and techniques are described in many standard laboratory manuals, such as R. Kingston, CURRENT PROTOCOLS IN MOLECULAR BIOLOGY, Chapter 9 (Sections I-IV) (2003)(DOI: 10.1002/0471142727.mb0900s64). See also, e.g. U.S. Pat. Nos. 4,419,446 and 4,601,978; Gray et al., Nature 295: 503-508 (1982); Canaani et al., Proc. Natl. Acad. Sci. USA 79: 5166-5170 (1982).

Transcription of DNA encoding a target enzyme or specific protein substrate may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at basepairs 100 to 270, the cytomegalovirus early promoter enhancer, the polyoma enhancer on the late side of the replication origin, and adenovirus enhancers. For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals. Construction of suitable vectors containing one or more of the above listed components employs standard ligation techniques. Isolated plasmids or DNA fragments are cleaved, tailored, and religated in the form desired to generate the plasmids required.

The protein may be expressed in a modified form, such as a fusion protein (e.g. a GST-fusion), and may include not only secretion signals, but also additional heterologous functional regions. For instance, a region of additional amino acids, particularly charged amino acids, may be added to the N-terminus of the polypeptide to improve stability and persistence in the host cell. A fusion protein may comprise a heterologous region from immunoglobulin that is useful to solubilize proteins. In one embodiment, the method of the invention may employ an expression vector that comprises a first polynucleotide encoding a target enzyme operably linked to a second polynucleotide encoding its specific protein substrate, in order to express the enzyme and its substrate as a fusion protein.

Transient transfection of the cells, as presently disclosed, enables a high level of expression and activity of transfected genes (e.g. target post-translation modification enzymes and their specific protein substrates) relative to background (e.g. endogenous) expression and activity of those genes, thus allowing the detection of substantially non-endogenous enzyme activity upon the introduced substrate. Accordingly, cell lines utilized in the methods and kits of the invention should be selected so as not to exhibit high endogenous activity of target enzymes of interest.

The term “adherent” when used herein to describe the cell, refers to a cell which naturally adheres to the first solid phase (often the well of the first assay plate), thereby forming a fairly uniform coating of the cells on the inside surface of the well. The uniform coating of cells generally forms following incubation of the cells in the wells of the first assay plate for about 8-16 hours. After incubation, non-adhering cells and cell culture medium are decanted off the first assay plate. Incubation is usually carried out at a temperature which is optimal for cell growth, i.e., about 37 degrees C. The cells to be added to the wells of the first assay plate may be maintained in tissue culture flasks and utilized when cells densities of about 70-90% of confluency are achieved. Often, the cells are diluted in culture medium prior to seeding them in the wells of the microtiter plate to achieve the desired cell densities.

The mammalian host cells used to transiently express the target post-translational modification enzyme(s) and specific protein substrate(s) may be cultured in a variety of standard media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Methods in Enzymology 58:4 4 (1979), Barnes et al., Anal. Biochem. 102: 255 (1980), U.S. Pat. Nos. 4,767,704; 4,657,866; 4,927,762; or 4,560,655; WO 90/03430; WO 87/00195; or U.S. Pat. No. 5,122,469, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. In general, principles, protocols, and practical techniques for maximizing the productivity of mammalian cell cultures can be found in MAMMALIAN CELL BIOTECHNOLOGY: A PRACTICAL APPROACH, M. Butler, ed., IRL Press (1991).

Gene amplification and/or expression may be measured in a sample directly, for example, by conventional Southern blotting, Northern blotting to quantitate the transcription of mRNA (Thomas, Proc. Natl. Acad. Sci. USA 77: 5201-5205 (1980)), dot blotting (DNA analysis), or in situ hybridization, using an appropriately labeled probe, based on the sequences provided herein.

Gene expression, alternatively, may be measured by immunological methods, such as immunohistochemical (IHC) staining to quantitate directly the expression of gene product.

“Antibody” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

Polyclonal antibodies useful in the practice of the invention may be produced according to standard techniques by immunizing a suitable animal (e.g., rabbit, goat, etc.) with an antigen encompassing a desired post-translational modification, collecting immune serum from the animal, and separating the polyclonal antibodies from the immune serum, in accordance with known procedures. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

Monoclonal antibodies useful in the practice of the invention may be produced in a hybridoma cell line according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

Antibodies useful in the practice of the invention, whether polyclonal or monoclonal, may be screened for epitope and post-translational modification-specificity according to standard techniques. See, e.g. Czemik et al., Methods in Enzymology, 201: 264-283 (1991). For example, the antibodies may be screened against a phospho and non-phospho peptide library by ELISA to ensure specificity for both the desired antigen, and for reactivity only with the phosphorylated form of the antigen. Peptide competition assays may be carried out to confirm lack of reactivity with other non-target phospho-epitopes. The antibodies may also be tested by Western blotting against cell preparations containing target protein, e.g. cell lines over-expressing it, to confirm reactivity with the desired phosphorylated target. Specificity against the desired phosphorylated epitopes may also be examined by construction target protein mutants lacking phosphorylatable residues at positions outside the desired epitope known to be phosphorylated, or by mutating the desired phospho-epitope and confirming lack of reactivity. Antibodies may be further characterized via immuno-histochemical (IHC) staining using normal and diseased tissues to examine target protein phosphorylation and activation status in diseased tissue.

A vast array of suitable capture and detection antibodies (including post-translational modification site- and residue-specific antibodies) are presently commercially available for purchase from a number of well-known vendors, including Cell Signaling Technology, Inc., Millipore/Upstate, Epitomics, Inc., Abcam, Inc., and Santa Cruz Biotechnology, Inc. This broad commercial selection allows for the off-the-shelf purchase of phospho-specific, and general, antibodies specific for most, if not all, of the currently known kinases (as well as other post-translational modification enzymes) and many of their substrates.

Other selective detection antibodies, such as streptavidin, which binds selectively to the “strep-tag” polypeptide, can also be employed (see Schmidt et al., Protein Engineering 6(1):109-122 (1993)). Streptavidin can be purchased commercially from a number of vendors, for example Zymed Laboratories.

The capture antibody binds specifically to a capture tag (which is present in the specific protein substrate transiently expressed by the transfected cells). Non-limiting examples of suitable capture tags and their respective capture antibodies include the flu HA flag and its antibody 12CA5, (Field et al., Mol. Cell. Biol. 8: 2159-2165 (1988)); the c-myc flag and the 8F9, 3C7, 6E10, G4, B7 and 9E10 antibodies thereto (Evan et al., Molecular and Cellular Biology 5(12): 3610-3616 (19850); as well as the Herpes Simplex virus glycoprotein D (gD) flag and the 5B6 antibody thereto (Paborsky et al., Protein Engineering 3(6): 547-553 (1990) and Mark et al., Journal of Biological Chemistry 269(14):10720-10728 (1994). Other suitable capture tags have been described. Examples include the Flag-peptide (Hopp et al., BioTechnology 6:1204-1210 (1988)); the KT3 epitope peptide (Martin et al., Science 255:192-194 (1992)); an alpha-tubulin epitope peptide (Skinner et al., J Biol. Chem 266:15163-15166 (1991)); and the T7 gene 10 protein peptide tag (Lutz-Freyermuth et al., Proc. Natl. Acad. Sci. USA 87: 6393-6397 (1990)). Once the capture tag has been selected as discussed above, a capture antibody thereto can be generated using the techniques disclosed herein.

Similarly, a broad array of commercially available organic fluorophores and quantum dots commercially suitable as detectable labels in the disclosed methods and kits to enable fluorescent imaging of captured target substrate(s). For example, several hundred different functionalized fluorophores and quantum dots are commercially available from Invitrogen (Carlsbad, Calif.), Sigma-Aldrich (St. Louis, Mo.), Biotium (Hayward, Calif.), Dyomics, GmbH (Jena, Germany), Thermo Fischer Scientific (Waltham, Mass.), and Interchim (San Diego, Calif.), among others.

Suitable quantum dots may include dots made from cadmium mercury telluride (CdHgTe), cadmium selenide (CdSe), cadmium selenide/zinc sulfide (CdSe/ZnS), cadmium sulfide (CdS), cadmium telluride (CdTe), cadmium telluride/cadmium sulfide (CdTe/CdS), and cadmium-free quantum dots, such as lead selenide (PbSe), lead sulfide (PbS), copper indium sulfide (CuInS), copper indium sulfide/zinc sulfide (CuInS/ZnS), or the like. The choice of which colors of quantum dots to select (typically varying based on size for a particular quantum dot composition) and which type(s) of bond(s) one wishes to make between the functionalized quantum dot and the detectable antibody can be made based on the level of multiplex (e.g. number of target enzymes and specific protein substrates) being employed in the assay. In a certain embodiment, the quantum dots may comprise quantum dots having an emission wavelength of about 450 nanometers to about 850 nanometers, or a wavelength selected from the group consisting of 525 nanometers, 545 nanometers, 565 nanometers, 585 nanometers, 605 nanometers, 625 nanometers, 655 nanometers, 705 nanometers, and 800 nanometers.

Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores, many of which are proprietary, often perform better (more photostable, brighter, and/or less pH-sensitive) than traditional dyes with comparable excitation and emission.

Common generic dye families that may be employed as detectable labels in the disclosed methods include Xanthene derivatives (e.g. fluorescein, rhodamine, OREGON GREEN, eosin, TEXAS RED, CAL FLUOR dyes, eosines, phloxines, uranines, succineins, sacchareins, rosamines, rhodols, pyranines, anthraquinones, benzopyrans, thioxanthenes, perylene imides, phenanthridines, carbopyronins, and fluorescent proteins such as green fluorescent protein and yellow fluorescent protein); Cyanine derivatives (e.g. cyanine, indocarbocyanine, oxacarbocyanine, thiacarbocyanine, merocyanine, and QUASAR dyes); Naphthalene derivatives (e.g. dansyl and prodan derivatives); Coumarin derivatives; Oxadiazole derivatives (e.g. pyridyloxazole, nitrobenzoxadiazole and benzoxadiazole); Pyrene derivatives (e.g. CASCADE BLUE, etc.); Oxazine derivatives (e.g. Nile red, Nile blue, cresyl violet, oxazine 170, etc.); Acridine derivatives (e.g. proflavin, acridine orange, acridine yellow, etc.); Arylmethine derivatives (e.g. auramine, crystal violet, malachite green); and Tetrapyrrole derivatives (e.g. porphin, phtalocyanine, bilirubin), among others.

Common proprietary/trademarked dye families may include CYDYE (GE Healthcare); CF dye (Biotium); BODIPY (Invitrogen); ALEXA FLUOR (Invitrogen); DYLIGHT FLUOR (Thermo Scientific, Pierce); ATTO and TRACY (Sigma Aldrich); FLUOPROBES (Interchim); MEGASTOKES Dyes (Dyomics); SETA Dyes (SETA BioMedicals); SETAU Dyes (SETA BioMedicals); and SQUARE Dyes (SETA BioMedicals), among others.

Detectable antibodies employed in the invention may likewise be conjugated to other, common non-fluorescent detectable groups such as radiolabels (e.g., S³⁵, I¹²⁵, I¹³¹), enzyme labels (e.g., horseradish peroxidase, alkaline phosphatase), and the like in accordance with known techniques.

Another embodiment of the invention provides a multiplex kit for assaying the effect of a test compound on the activity of multiple kinases in a biological cell, the kit comprising:

(a) a plurality of human embryonic kidney (HEK) 293 cells transiently transfected with one or more expression vectors comprising a plurality of polynucleotides encoding (i) a plurality of target kinases and (ii) a plurality of specific protein substrates of the target kinases, wherein each specific protein substrate is linked to at least one unique capture tag that is not conjugated to a rare earth element;

(b) one or more multi-well capture plates or beads comprising a plurality of capture antibodies, each of which specifically binds to one of the unique capture tags linked to a specific protein substrate;

(c) optionally, a plurality of human embryonic kidney (HEK) 293 cells transiently transfected with one or more expression vectors comprising a plurality of polynucleotides encoding (i) a plurality of target inactive mutant kinases and (ii) a plurality of specific protein substrates of the target kinases, wherein each specific protein substrate is linked to at least one unique capture tag that is not conjugated to a rare earth element, and wherein the expression vectors are suitable for transiently transfecting the cells;

(d) optionally, one or more reagents for culturing and/or lysing the cells transiently transfected with the expression vectors;

(e) optionally, a plurality of labeled detection antibodies each of which specifically binds to the phosphorylated form of a specific protein substrate but does not substantially bind to the un-phosphorylated form of that specific protein substrate

(f) optionally, one or more control compounds for inhibiting one or more of the target kinases; and

(g) instructions for (i) culturing the transfected cells under conditions suitable to transiently express the plurality of target kinases and the plurality of specific protein substrates, (ii) plating the cells in multi-well plates, contacting the cells with at least one test compound, and incubating the cells under conditions suitable to allow phosphorylation of the specific protein substrates by the target kinases, (iii) lysing the cells, transferring the lysed cells to the multi-well capture plate of step (b), and capturing the plurality of specific protein substrates on or in the multi-well capture plate, (iv) detecting the presence of phosphorylated forms of the plurality of specific protein substrates using the plurality of labeled detection antibodies; and (v) comparing the level of phosphorylated forms of the plurality of specific protein substrates detected in a test sample of cells contacted with the test compound with 1) the level of phosphorylated forms of the plurality of specific protein substrates in at least one control sample taken from control HEK 293 cells not contacted with the test compound, and 2) the level of phosphorylated forms of the plurality of specific protein substrates in at least one control sample taken from control HEK 293 cells not contacted with the test compound but rather treated with known target kinase inhibitor(s), or otherwise equivalent cells expressing kinase-deficient mutant instead of active forms of the target kinases, thereby assaying the effect of the test compound on the activity of the plurality of target kinases within the cells contacted with the test compound.

In one embodiment, a multiplex kit is provided wherein the activities of the plurality of target kinases that are assayed are substantially non-endogenous kinase activities. In another embodiment of the multiplex kit, the plurality of target kinases comprise at least one tyrosine kinase and at least one serine/threonine kinase. In yet another embodiment of the multiplex kit, the first and second capture tags are selected from the group consisting of FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, and Glu-Glu, or combinations thereof. In still another embodiment of the kit, the plurality of capture antibodies of step (b) are bound to the multi-well capture plate, while in yet another embodiment, the plurality of capture antibodies of step (b) are conjugated to beads that are not bound to the multi-well capture plate.

In another embodiment, there is provided a multiplex kit for assaying the effect of a test compound on the activity of multiple post-translational modification enzymes in a biological cell, the kit comprising:

(a) a plurality of human embryonic kidney (HEK) 293 cells transiently transfected with one or more expression vectors comprising a plurality of polynucleotides encoding (i) a plurality of target post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells;

(b) one or more multi-well capture plates or beads comprising a plurality of capture antibodies, each of which specifically binds to one of the unique capture tags linked to a specific protein substrate;

(c) optionally, a plurality of human embryonic kidney (HEK) 293 cells transiently transfected with one or more expression vectors comprising a plurality of polynucleotides encoding (i) a plurality of target inactive mutant post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells;

(d) optionally, one or more reagents for culturing and/or lysing the cells transiently transfected with the expression vectors;

(e) optionally, a plurality of labeled detection antibodies each of which specifically binds to the post-translationally modified form of a specific protein substrate but does not substantially bind to the unmodified form of that specific protein substrate

(f) optionally, one or more control compounds for inhibiting the target post-translational modification enzymes; and

(g) instructions for (i) culturing the transfected cells under conditions suitable to transiently express the plurality of target post-translational modification enzymes and the plurality of specific protein substrates, (ii) plating the cells in multi-well plates, contacting the cells with at least one test compound, and incubating the cells under conditions suitable to allow post-translational modification of the specific protein substrates by the target post-translational modification enzymes, (iii) lysing the cells, transferring the lysed cells to the multi-well capture plate, and capturing the plurality of specific protein substrates on or in the multi-well capture plate, (iv) detecting the presence of post-translationally modified forms of the plurality of specific protein substrates using the plurality of labeled detection antibodies; and (v) comparing the level of post-translationally modified forms of the plurality of specific protein substrates detected in a test sample of cells contacted with the test compound with 1) the level of post-translationally modified forms of the plurality of specific protein substrates in at least one control sample taken from control HEK 293 cells not contacted with the test compound, and 2) the level of post-translationally modified forms of the plurality of specific protein substrates in at least one control sample taken from control HEK 293 cells not contacted with the test compound but rather treated with known target post-translational modification enzyme inhibitor(s), or otherwise equivalent cells expressing enzyme activity-deficient mutant instead of active forms of the target post-translational modification enzymes, thereby assaying the effect of the test compound on the activity of the plurality of post-translational modification enzymes within the cells contacted with the test compound.

In one embodiment of the multiplex kit, the activities of the plurality of post-translational modification enzymes that are assayed are substantially non-endogenous post-translational modification enzyme activities. In another embodiment of the multiplex kit, the first and second capture tags are selected from the group consisting of FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, and Glu-Glu, or combinations thereof. In another embodiment of the multiplex kit, the plurality of capture antibodies of step (b) are bound to the multi-well capture plate, while in another, the plurality of capture antibodies of step (b) are conjugated to beads that are not bound to the multi-well capture plate.

In still another embodiment of the multiplex kit, at least one target post-translational modification enzyme is a methylase and at least one post-translational modification is methylation. In still another, at least one target post-translational modification enzyme is an acetylase and at least one post-translational modification is acetylation, while in yet another, at least one target post-translational modification enzyme is a glycosylase and at least one post-translational modification is glycosylation. In yet still another embodiment of the multiplex kit, at least one target post-translational modification enzyme is an enzyme selected from Column 1, Table 1 (above), and wherein at least one corresponding post-translational modification is selected from the corresponding row of Column 2, Table 1.

The kit may also include one or more secondary reagents, such as a secondary antibody, or ancillary agents such as buffering agents and protein stabilizing agents, e.g., polysaccharides and the like. The kit may further include, where necessary, other enzyme substrates, agents for reducing background interference in a test, control reagents, apparatus for conducting a test, and the like.

Where the detectable label is an enzyme, the kit may further include substrates and cofactors required by the enzyme (e.g. a substrate precursor which provides the detectable chromophore or fluorophore). Conveniently, the kit can also supply the homogeneous population of transiently transfected cells (or transfectable cells together with expression vectors and reagents suitable for transiently transfecting the cells) which transiently express desired target enzyme(s) and specific protein substrates. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration. The kit also suitably includes instructions for carrying out the assay.

In still another embodiment, there is provided a method for assaying the effect of a test compound on the activity of a post-translational modification enzyme in a biological cell, the method comprising the steps of:

(a) obtaining a plurality of transiently transfected biological cells that transiently express (i) at least one target post-translational modification enzyme and (ii) at least one specific protein substrate of the target post-translational modification enzyme, wherein the specific protein substrate is linked to at least one capture tag that is not conjugated to a rare earth element;

(b) culturing the plurality of biological cells under conditions suitable to transiently express the target post-translational modification enzyme and the specific protein substrate;

(c) incubating the plurality of biological cells with at least one test compound under conditions suitable to allow post-translational modification of the specific protein substrate by the target post-translational modification enzyme;

(d) lysing the plurality of biological cells and capturing the specific protein substrate using at least one capture antibody that specifically binds the capture tag;

(e) detecting the presence of post-translationally modified specific protein substrate using at least one detection antibody that specifically binds the post-translationally-modified form of the specific protein substrate but does not substantially bind the specific protein substrate when not post-translationally modified; and

(f) comparing the level of post-translationally modified specific protein substrate detected in step (e) with 1) the level of post-translationally modified specific protein substrate in at least one control sample taken from control biological cells not contacted with the test compound, and 2) the level of post-translationally modified specific protein substrate in at least one control sample taken from control biological cells not contacted with the test compound but rather treated with known target post-translational modification enzyme inhibitor(s), or otherwise equivalent cells expressing enzyme activity-deficient mutant instead of active forms of the target post-translational modification enzymes, thereby assaying the effect of the test compound on the activity of the target post-translational modification enzyme within the biological cells of step (c).

In practicing the disclosed assay methods, washing is typically employed by exposing the solid phase to an aqueous solution (usually a buffer or cell culture media) in such a way that unbound material (e.g., non-adhering cells, non-adhering capture agent, unbound ligand, receptor, receptor construct, cell lysate, or anti-phosphotyrosine antibody) is removed therefrom. To reduce background noise, it is convenient to include a detergent (e.g. Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) may be required.

Block buffer—an aqueous, pH buffered solution containing at least one blocking compound which is able to bind to exposed surfaces of the second solid phase which are not coated with capture antibody—may also typically be employed. The blocking compound is normally a protein such as bovine serum albumin (BSA), gelatin, casein or milk powder and does not cross-react with any of the reagents in the assay (e.g., an anti-phosphoserine antibody and detection reagents). The block buffer is generally provided at a pH between about 7 to 7.5 and suitable buffering agents include phosphate and TRIS.

Similarly, the practice of the disclosed methods may usefully employ lysis buffer, an aqueous, pH buffered solution comprising a solubilizing detergent, one or more protease inhibitors and at least one phosphatase inhibitor (such as sodium orthovanadate). The term “solubilizing detergent” refers to a water miscible, non-ionic detergent which lyses cell membranes of eukaryotic cells but does not denature or activate the receptor or receptor construct. Examples of suitable non-ionic detergents include Triton-X 100, Tween 20, CHAPS and Nonidet P-40 (NP40) available from Calbiochem, La Jolla, Calif., for example. Many other non-ionic detergents are available in the art. Examples of suitable protease inhibitors include phenylmethylsulfonyl fluoride (PMSF), leupeptin, pepstatin, aprotinin, 4-(2-aminoethyl)-benzenesulfonyl fluoride hydrochloride-bestatin, chymostatin and benzamidine. Preservatives (e.g., thimerosal) and one or more compounds which maintain the isotonicity of the solution (e.g., sodium chloride (NaCl) or sucrose) and a buffer (e.g., Tris or PBS) are usually also present. Generally, the pH of the lysis buffer is in the range about 7 to 7.5.

Usually, following addition of the lysis buffer to the first assay plate, the first assay plate is “gently agitated” and this expression refers to the act of physically shaking the first assay plate (normally using a circular motion) at a substantially low velocity. Gentle agitation does not involve mechanically disrupting the cells (e.g. by homogenizing or centrifuging the cells). Exemplary shaking velocities are in the order of 200 to 500 rpm, preferably 300 to 400 rpm in a Bellco orbital shaker, for example.

The test compound(s) is/are added to each well that contains the adhering cells using a pipette, for example. At least one control well (e.g. to which the aqueous diluent for the test compound is added) is included in the assay. The adhering cells are usually contacted for a sufficient period of time to allow modulation (e.g. inhibition) of the target enzyme activity on the specific protein substrate by the test compound, but not too long such that the signal decreases as a consequence of dephosphorylation of the modified substrates by endogenous phosphatases. A suitable incubation period is typically between about 1 to 3 hours, preferably about 2 hours at a physiologically optimal temperature for the cells (usually about 37 degrees C.). Following incubation, cells are lysed and assessed directly for the level of phosphorylated exogenous substrate.

In an embodiment of the method, the specific protein substrate is not a plurality of promiscuous peptide substrates capable of being post-translationally modified by a plurality of post-translational modification enzymes. In another embodiment, the plurality of post-translational modification enzymes are kinases and wherein the promiscuous peptide substrates are capable of being phosphorylated by the plurality of kinases. In another embodiment, the target post-translational modification enzyme is a tyrosine kinase and wherein the post-translational modification is phosphorylation. In still another embodiment of the method, the specific protein substrate is a domain of the tyrosine kinase that is auto-phosphorylated by the kinase domain of the tyrosine kinase. In yet another embodiment, the biological cells of step (a) have been transiently transfected with at least one polynucleotide comprising a polynucleotide encoding the tyrosine kinase.

In another embodiment of the method, the target post-translational modification enzyme is a serine/threonine kinase and wherein the post-translational modification is phosphorylation. In another embodiment, the specific protein substrate is (i) a domain of the serine/threonine kinase that is auto-phosphorylated by the kinase domain of the serine/threonine kinase or (ii) not a domain of the serine/threonine kinase. In still another embodiment, the biological cells of step (a) have been transiently co-transfected with at least one first polynucleotide comprising a polynucleotide encoding the serine/threonine kinase and at least one second polynucleotide comprising a polynucleotide encoding the specific protein substrate. In yet another embodiment of the method, the biological cells of step (a) have been serum starved prior to co-transfection.

In a further embodiment of the method, the first and second polynucleotides are comprised within a single expression vector, while in another embodiment, the first and second polynucleotides are operably linked to express a fusion protein comprising the serine/threonine kinase and the specific protein substrate. In still another embodiment, the transiently transfected biological cells of step (a) are capable of expressing the target post-translational modification enzyme and the specific protein substrate within about 1-5 days of being transfected, and wherein the transiently transfected biological cells are not stably transformed and do not stably express the target post-translational modification enzyme or the specific protein substrate, other than in endogenous form.

In still yet another embodiment of the method, the capture tag is selected from the group consisting of FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, Glu-Glu, or combinations thereof.

In another embodiment, the method is employed in multiplex fashion utilizing two or more different, target post-translational modification enzymes and two or more different, specific protein substrates to assay the effect of at least one test compound on the activity of the two or more different, target post-translational modification enzymes. In an embodiment, the two or more different target post-translational modification enzymes are tyrosine kinases, serine/threonine kinases, or combinations thereof, and wherein the post-translational modification is phosphorylation. In another embodiment, the biological cells of step (a) have been transiently transfected with (i) at least one first polynucleotide comprising a polynucleotide encoding a tyrosine kinase, (ii) at least one second polynucleotide comprising a polynucleotide encoding a serine/threonine kinase, and optionally (iii) at least one third polynucleotide comprising a polynucleotide encoding the specific protein substrate of the serine/threonine kinase.

In yet another embodiment of the method, step (c) is carried out in a multi-well plate, while in another embodiment, step (d) further comprises transferring the lysed biological cells to a multi-well plate, and wherein the capture antibody is affixed to a plurality of wells in the plate or to a plurality of beads added to the plate. In still another embodiment, the biological cells of step (a) comprise human primary cells, human stem cells, human cell lines, or combinations thereof.

In still another embodiment of the method, the detection antibody of step (e) comprises a detectable label. In one embodiment, the detectable label is selected from the group consisting of horse radish peroxidase, quantum dots, fluorophores, alkaline phosphatase, or combinations thereof. In another embodiment, the detection antibody of step (e) is a primary antibody that is specifically bound by a secondary antibody, wherein the secondary antibody comprises a detectable label or is bound to another molecule that comprises one or more detectable labels. In yet another embodiment, the detection antibody of step (e) is a phospho-tyrosine specific antibody. In still another embodiment of the method, the detection antibody of step (e) is a phosphorylation-site specific antibody that specifically binds the specific protein substrate when phosphorylated at a particular serine or threonine site but does not substantially bind the specific protein substrate when not phosphorylated at that particular serine or threonine site. In still yet another embodiment, the specific protein substrate is BAD and wherein the phosphorylation-site specific antibody is phospho-BAD (serine 112) antibody.

Additional examples of suitable enzymatic detectable labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating such label enzymes to antibodies are described in, e.g. O'Sullivan et al., Methods in Enzymology 73: 147-166 (J. Langone & H. Van Vunakis eds.), Academic Press, New York, (1981).

Further examples of enzyme-substrate combinations suitable for use as detectable labels in the presently disclosed methods and kits include: (i) Horseradish peroxidase (HRPO) with hydrogen peroxidase as a substrate, wherein the hydrogen peroxidase oxidizes a dye precursor (e.g. orthophenylene diamine (OPDD or 3,3′5,5′-tetramethyl benzidine hydrochloride (TMB)); (ii) alkaline phosphatase (AP) with para-Nitrophenyl phosphate as chromogenic substrate; and (iii) beta-D-galactosidase with a chromogenic substrate (e.g. p-nitrophenyl-beta-D-galactosidase) or fluorogenic substrate 4-methylumbelliferyl-beta-D-galactosidase. Numerous other enzyme-substrate combinations are available to those skilled in the art. For a general review of these, see U.S. Pat. Nos. 4,275,149 and 4,318,980.

In a further embodiment of the method, the tyrosine kinase is a receptor tyrosine kinase selected from the group consisting of an ALK, AXL, DDR, EGFR, EPH, FGFR, INSR, MET, MUSK, PDGFR, PTK7, RET, ROR, ROS, RYK, TIE, TRK, VEGFR, and AATYK family kinase. In another embodiment, the tyrosine kinase is a cytoplasmic tyrosine kinase selected from the group consisting of an ABL, ACK, CSK, FAK, FES, FRK, JAK, SRC, TEC, or SYK family kinase. In still another embodiment, the tyrosine kinase or serine/threonine kinase is a tyrosine kinase like (TKL) kinase selected from the group consisting of an MLK, RAF, STKR, LRRK, LISK, IRAK, and RIPK family kinase. In yet another embodiment of the method, the serine/threonine kinase is an AGC family kinase selected from the group consisting of a AktR, Aid, GRK, MAST, DMPK, NDR, PDK1, PKA, PKC, PKG, PKN, RSK, RSKL, RSKR, SGK, AGC-Unique, YANK, AGC-Sar, AGC1, AGC, and PTF kinase.

In yet another embodiment of the method, the serine/threonine kinase is a CAMK family kinase selected from the group consisting of a CDPK, CAMKL, CAMK1, CAMK2, DAPK, DCAMKL, MAPKAPK, MLCK, Trb1, PHK, PKD, PSK, RSKb, CAMK-Unique, CAMK-Tt, CASK, PIM, RAD53, Trio, TSSK, Ciliate-C1, STK33, SgK495, CAMKX, CAMKY, Chk1L, MLCK-like, PPCK, CAMK-Tvag1, CAMK-Tvag2, and CAMK-Tvag3 kinase.

In yet still another embodiment of the method, the serine/threonine kinase is a CK1 family kinase selected from the group consisting of a CK1, Dual, TTBK, Worm10, Worm11, Worm7, Worm8, Worm9, TTBKL, VRK, and CK1-Unique kinase. In another embodiment, the serine/threonine kinase is a CMGC family kinase selected from the group consisting of a Dicty1, CLK, CK2, CDK, CDKL, DYRK, GSK, MAPK, SRPK, CMGC-Unique, RCK, CMGC-GL1, and CMGC-Tvag1 kinase. In still another embodiment, the serine/threonine kinase is a STE family kinase selected from the group consisting of a Dicty2, Dicty3, STET, STE11, STE20, Ste-Unique, and STE-plant1 kinase. In still another embodiment of the method, the serine/threonine kinase is a receptor guanylate cyclase (RGC) family kinase.

In a further embodiment of the method, the serine/threonine kinase is an Atypical family kinase selected from the group consisting of a AFK, A6, BCR, BRD, HisK, PDHK, TIF1, FAST, G11, TAF1, H11, and PI3 kinase. In yet another embodiment, the serine/threonine kinase is selected from the group consisting of a Worm6, Dicty10, Dicty6, Dicty7, Dicty8, Dicty9, Aur, CAMKK, CDC7, urch, WEE, IKK, IRE, IKS, NEK, Slob, NKF1, NKF2, NKF3, NKF4, NKF5, NRBP, NAK, PLK, SAMK, TBCK, Worm1, Worm2, Worm3, Worm4, Worm5, Other-Unique, TLK, Ciliate-A1, Ciliate-A2, Ciliate-A3, Ciliate-A4, Ciliate-A5, Ciliate-A6, Ciliate-A7, Ciliate-A8, Ciliate-A9, Ciliate-B1, Ciliate-B2, Ciliate-B3, Ciliate-C8, Ciliate-C3, Ciliate-C4, Ciliate-C5, Ciliate-C6, Ciliate-C7, Ciliate-D1, Ciliate-E1, Ciliate-E2, Ciliate-E3, Ciliate-E4, Ciliate-E5, Ciliate-E6, Ciliate-E7, Ciliate-E8, Ciliate-E9, Ciliate-F1, Ciliate-F2, WNK, Bub, Bud32, HAL, Haspin, MOS, PEK, RAN, SCY1, TOPK, TTK, ULK, VPS15, Ciliate-G, Ciliate-Unique, Ciliate_C8_Cterm, Dusty, KIS, SgK071, SgK493, SgK496, Unil, AgaK1, FunK1, HisK, Mak2, Mbre3, NEK-frag, NEK-like, Other-GL1, Other-GL2, Other-Tvag1, Other-Tvag3, and Other-Tvag2 kinase.

The above mentioned kinases, their full names, activity, and substrates, are well known and described in the art. Additional kinase information can be readily found at the PhosphoSite database, which includes protein and gene sequence information for each kinase. Additional nucleic acid sequence sources for kinases are well known in the art, and include, e.g. GenBank and NCBI.

In another embodiment of the disclosed method, the target post-translational modification enzyme is a methylase and the post-translational modification is methylation, while in another embodiment, the target post-translational modification enzyme is an acetylase and the post-translational modification is acetylation. In yet another embodiment, the target post-translational modification enzyme is a glycosylase and the post-translational modification is glycosylation. In still another embodiment of the method, the target post-translational modification enzyme is an enzyme selected from Column 1, Table 1, and wherein the corresponding post-translational modification is selected from the corresponding row of Column 2, Table 1. In still another embodiment, the method of claim 38, wherein the activity of the post-translational modification enzyme that is assayed is substantially non-endogenous enzyme activity.

The teachings of all references cited herein are hereby incorporated herein in their entirety by reference. The following Examples are provided to further illustrate the disclosed methods and kits, but do not limit the disclosed invention except as described in the claims.

All publications, patents and patent applications cited herein, whether supra or infra, are hereby incorporated by reference in their entirety. As used in this specification and the appended claims, the singular forms “a,” “an,” and “the” include plural references unless the content clearly dictates otherwise. The terms used in this invention adhere to standard definitions generally accepted by those having ordinary skill in the art. In case any further explanation might be needed, some terms have been further elucidated below.

“Antibody” is used in its broadest sense, and includes monoclonal, polyclonal, recombinant, and/or chimeric antibodies, or antibody fragments, having a desired binding affinity.

“Capture antibody” means an antibody capable of specifically binding to its corresponding capture tag, thus allowing the specific separation or capture of a target protein bearing the capture tag from other proteins that do not have the capture tag.

“Capture tag” means any tag, label, or flag linked or bound to a target protein that allows for the specific separation or capture of that target protein from other proteins that do not have the capture tag.

“Detectable antibody” means an antibody that comprises at least one detectable label.

“Detection antibody” means an antibody capable of specifically binding to a target protein, thus allowing the specific detection of that target protein from other non-target proteins. In the case of post-translationally modified target protein, the detection antibody may be an antibody that specifically binds the target protein only when modified at particular residues (e.g. phospho-tyrosine, acetyl-lysine) or only when modified at a particular site/epitope (e.g. phospho-serine at position 273).

“Detectable label” means any tag, label, or flag (including, but not limited to dyes, fluorophores, radio-isotopes, etc.), that may be suitably affixed, linked, or bound to a detection antibody and is capable of either direct or indirect detection (by any suitable means, including, but not limited to, visualization, scintillation, fluorescence, etc.) thereby allowing the identification of target protein bound by such detection antibody.

“Does not substantially bind” means, with respect to an antibody that specifically binds a particular post-translational modification (e.g. a phosphotyrosine residue-specific antibody, or a phosphorylation site-specific antibody), that the binding of such antibody to the unmodified form of target protein, as compared to the level of its binding to modified form, is not statistically substantial or significant.

“Independent non-domain substrate” means a protein substrate of a given post-translational modification enzyme that does not comprise a domain or region of the enzyme, and therefore cannot be auto-phosphorylated by the catalytic/kinase domain of the enzyme, in contrast to a domain comprised within the enzyme which is capable of being auto-phosphorylated.

“Multiplex” means, with respect to an assay method or kit, the ability to assay (and format for assaying) simultaneously more than one target analyte, and frequently several target analytes, by employing more than one capture antibody and/or more than one detection antibody.

“Operably linked” means, with respect to a polynucleotides that have been joined or linked to encode a fusion protein, that the polynucleotide encoding the first portion of the fusion protein and the polynucleotide encoding the second portion of the fusion protein have been joined or linked in such manner that, when translated by enzymes within a host cell, the fusion protein will be expressed and active.

“Promiscuous peptide substrates” means a plurality of short peptides (e.g. 10 to 50 amino acids long) that are designed to, and are capable of, serving as substrates for a plurality of different, post-translational modification enzymes, such that the peptides are generic and do not serve as specific protein substrates of any single enzyme. For example, promiscuous peptides substrates for the receptor tyrosine kinases EGFR, VEGFR, and IGFR would be phosphorylated by all three of these enzymes, and would not be a specific substrate for any one of the three enzymes.

“Singleplex” means, with respect to an assay method or kit, the ability to assay (and format for assaying) one target analyte by employing one capture antibody and/or one detection antibody.

“Specific protein substrate” means a unique and naturally occurring protein substrate of a given enzyme, which, in the case of a post-translational modification enzyme, is specifically modified by the enzyme at one or more particular sites on the substrate protein. The term does not include promiscuous peptide substrates or other generic/non-specific engineered substrates that are bound and modified by multiple post-translational modification enzymes in a substrate-non-specific manner.

“Substantially non-endogenous” means, with respect to protein/enzyme expression and activity in a biological cell, that the expression and activity of a protein/enzyme that is native and endogenous to that biological cell, when compared to the expression and activity of a protein/enzyme that is introduced into the cell by transient transfection and over-expressed, is not statistically significant or substantial. For example, the activity of a tyrosine kinase that is transiently transfected into a biological cell and over-expressed, when assayed, will be substantial whereas the endogenous activity of that kinase will not be substantial as compared to the transiently transfected enzyme activity.

“Transiently express” means a biological cell that only expresses an externally introduced gene for a limited period of time, typically 1-5 days, and has not been transformed (i.e. the introduced gene has not stably incorporated into the DNA of the host cell) and is not capable of stably expressing the introduced gene for longer periods of time. Typically the externally introduced gene is being transiently expressed directly from a vector with which the biological cell has been transiently transformed.

“Transiently transfected” means, with respect to a biological cell, that an expression vector encoding an externally introduced gene has been introduced into the cell in order to transiently express the introduced gene, but that the biological cell does not become stably transformed by the vector and does not stably express the introduced gene for longer periods of time.

### EXAMPLES

### Example 1

### lck-FLAG® Tyrosine Kinase Autophosphorlation Assay

This assay was developed to quantify the extent of autophosphorylation of lck, or lymphocyte specific kinase, in a high-throughput manner. Lck is a 56-kD protein within the src family of tyrosine kinases, and is involved in signaling in T-cells. As such, selective inhibition of lck is expected to have clinical relevance for immune suppression, inflammation, and the treatment of transplant rejection. In this example, FLAG-tagged lck is overexpressed in HEK293F cells, and autophosphorylation of lck is measured within the activation loop at the site equivalent to Y416 in src. This assay enables potency measurements of lck inhibitors, as quantitative measurements of lck autophosphorylation can be performed in the presence of differing amounts of inhibitor to generate dose response curves and EC50 values.

(i) Expression Vectors and Antibodies

Plasmid DNA encoding human full length lck, transcript variant 2, with a C-terminal FLAG (DDK) and myc tag, in a mammalian expression vector, was obtained from Origene. Kinase-deficient lck (K273M) was made by mutating the lysine at amino acid 273 to methionine by in vitro site-directed mutagenesis, using the QuikChange XL Site Directed Mutagenesis Kit (Agilent Technologies) under the manufacturer's recommendations. Sequencing was performed to verify that the DNA coding sequence was as expected. Mouse monoclonal anti-FLAG antibody, clone M2, was obtained from Sigma Aldrich. Polyclonal antibody to the autophosphorylation site in lck, which is equivalent to the Y416 site in the human family member src, was obtained from Cell Signaling Technology (Phospho-Src Family (Tyr416) Antibody). Secondary antibodies (Anti-Rabbit IgG, HRP-linked) were also obtained from Cell Signaling Technology. Polyclonal antibody to the N-terminus of lck was obtained from Abgent.

(ii) Transient Transfection of Mammalian Cells

Human embryonic kidney HEK293F cells (293F; Life Technologies) were seeded in 6-well plates and grown to a confluency of approximately 70-80% using the manufacturer's recommended medium supplemented with 10% Fetal Bovine Serum. 2 ug of plasmid DNA encoding human full length lck with a C-terminal FLAG (DDK) tag was transfected into each well of the 293F cells using Lipofectamine 2000 (Life Technologies) under the manufacturer's recommendations. Plasmid encoding lck K273M kinase-deficient mutant was also utilized to transfect cells in the same manner, for negative controls. ˜20 hours following transfection, cells were harvested and plated at a density of approximately 1×10⁴ cells per well in 96-well plates. Transfections were also performed in the same way utilizing larger culture vessels (eg. T75 flasks), but scaling the amount of transfection and DNA reagents appropriately.

(iii) Cell Incubation and Compound Treatment

Eight twofold serial dilutions were prepared for each compound in 100% DMSO in 96-well polypropylene plates. Compounds were then diluted in water to 20× final assay concentration and 12% DMSO, and were added to the cells in 96-well plates (20-fold dilution in tissue culture medium) for a final concentration of 1× compound and 0.6% DMSO. For control cells with no compound treatment, DMSO alone was added in a similar manner to achieve a final concentration of 0.6%. Following the addition of compound or DMSO controls, cells were incubated at 37° C. for 2 hours.

(iv) ELISA Autophosphorylation Assay

In preparation for the ELISA, 96-well flat bottom assay plates were coated with anti-FLAG antibody for 1 hour at room temperature or overnight at 4 degrees C., then blocked for 1 hr. with 1% BSA in TBST (TBST consists of 10 mM Tris, pH7.4, 150 mM NaCl and 0.1% Tween 20). Transiently transfected cells treated with compound plus untreated controls in 96-well plates were lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates were then transferred to the coated, blocked ELISA plate. Following 1 hour incubation with lysate, plates were washed 3× with TBST followed by incubation with Phospho-Src Family (Tyr416) primary antibody at 1:1000 dilution in TBST. Following 1 hour incubation with primary antibody, plates were washed 3× with TBST and incubated with a 1:2500 dilution of secondary antibody, HRP-linked anti-rabbit IgG, in TBST. Following 1 hour incubation, plates were washed 3× with TBST, and 1-Step Slow TMB-ELISA reagent (Thermo Scientific) was added. After 10 minute incubation with substrate, equal volume of 2M H₂SO₄ was added, and absorbance was read at 450 nm.

(v) Assay Validation

The lck-FLAG autophosphorylation assay was validated in the following way: 1) verification of wt lck and K273M lck expression in transiently transfected 293F cells, following immunoprecipitation (IP) with an anti-FLAG antibody and Western blotting, 2) verification of wt lck autophosphorylation at the equivalent site to Tyr416 in src, and much reduced levels of phosphorylation with K273M lck, in transiently transfected 293F cells following IP with an anti-FLAG antibody and Western blotting, 3) signal for wt lck over background of K273M lck in the ELISA assay using transiently transfected 293F cells and Phospho-Src Family Tyr416 as the detection antibody, and 4) a dose dependent reduction in signal for phosphorylated lck in the ELISA assay using transiently transfected 293F cells and Phospho-Src Family Tyr416 as the detection antibody, after treatment with a compound that is known to inhibit lck kinase activity, but not with a compound that does not inhibit lck.

(v.1) Verification of lck and K273M lck Expression in Transiently Transfected 293F Cells

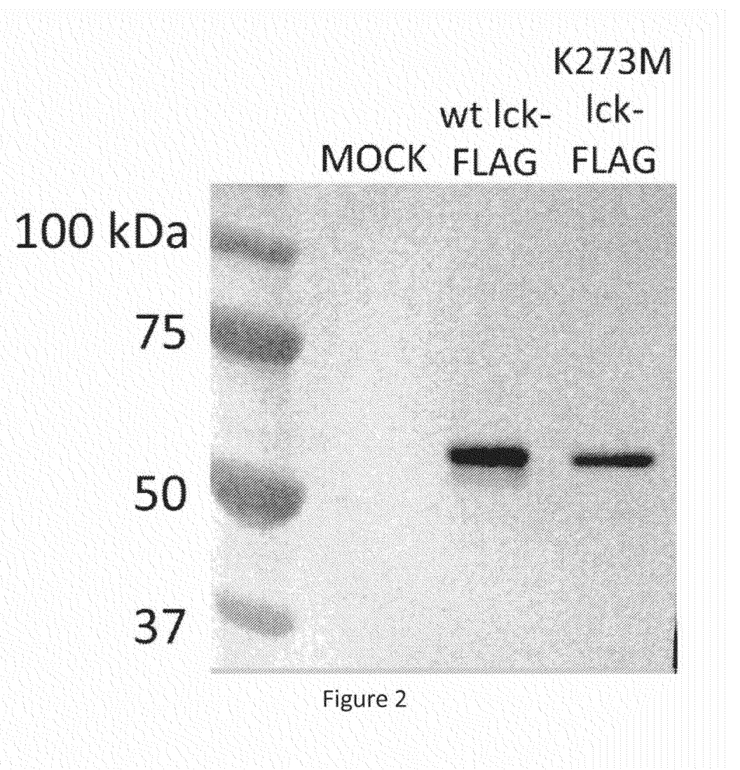

293F cells were transiently transfected in 6-well plates as outlined above, with 1) a vector expressing full length wild type human lck-FLAG, and 2) a vector expressing K273M kinase-deficient mutant full length human lck-FLAG. ˜20 hours after transfection, cells were harvested and lysed in 500 uL 1× lysis buffer per well. Anti-FLAG M2 Affinity Gel (Sigma Aldrich) was used to immunoprecipitate FLAG-tagged lck and lck 273M under the manufacturer's recommendations. Following IP, 100 uL of 2× gel sample buffer (Bio-Rad) was added and samples were boiled at 95 degrees C. for 3-5 minutes. 10 uL of each sample were run on an Any KD precast polyacrylamide gel (Bio-Rad), followed by transfer to nitrocellulose membrane. Membranes were blocked with 3% BSA in TBST for 1 hour, then probed with 1:500 anti-(total) lck antibody at 4 degrees C. overnight. Following washing 3× with TBST, membranes were probed with HRP-linked anti-rabbit IgG for 1 hour at room temperature. Following washing 3× with TBST, membranes were exposed to the chromogenic HRP Western blotting substrate mixture, CN/DAB (CN/DAB Substrate Kit; Thermo Scientific). Following development of the precipitate, membranes were photographed. The results of such analysis are shown in FIG. 2. FIG. 2 shows the expression of lck-FLAG and K273M lck-FLAG in transiently transfected 293F cells. Cells were transfected with the respective constructs, then IP was performed with anti-FLAG antibody followed by Western Blotting with anti-lck. Mock transfected cells (no DNA) were used as controls.

These data establish that both the lck-FLAG and K273M lck-FLAG are expressed in the 293F cells and are able to be immunoprecipitated with the M2 anti-FLAG antibody.

(v.2) Verification of lck Phosphorylation Over Background of K273M lck Phosphorylation in Transiently Transfected 293F Cells

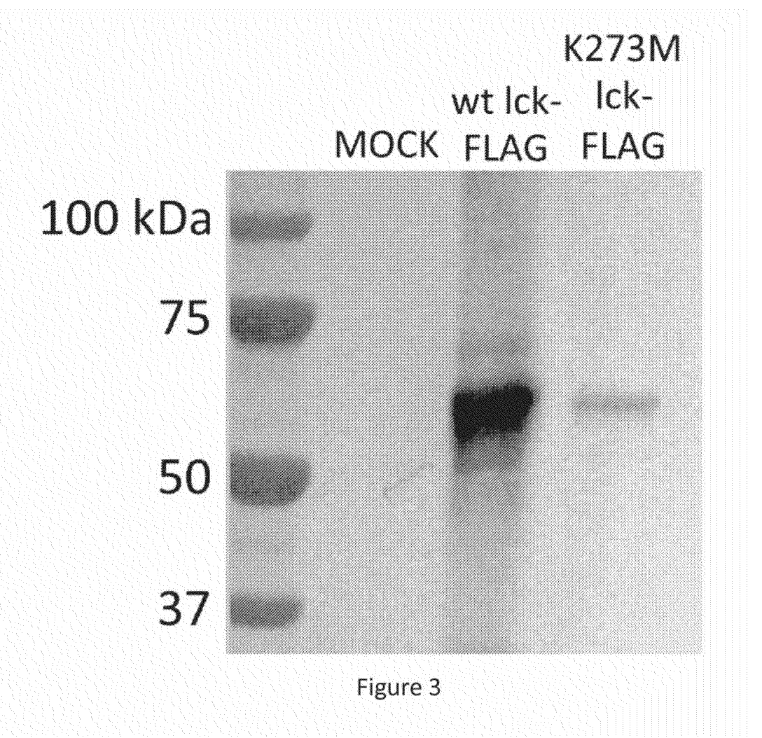

293F cells were transiently transfected followed by anti-FLAG immunoprecipitation as outlined above. Western blotting was then performed as above except that anti Phospho-Src Family (Tyr416) was utilized as the primary antibody. Results from this analysis are presented in FIG. 3. FIG. 3 shows phosphorylation of lck-FLAG over background of K273M lck-FLAG in transiently transfected 293F cells. Cells were transfected with the respective constructs, then IP was performed with anti-FLAG antibody followed by Western Blotting with anti-Phospho-Src Family (Tyr416). Mock transfected cells (no DNA) were used as controls.

These data establish that the lck-FLAG protein is phosphorylated at the site equivalent of Tyr416 in src, presumably by autophosphorylation, at a much higher level compared to the K273M kinase deficient mutant lck-FLAG.

(v.3) ELISA Signal Over Background Measuring lck Phosphorylation

Cells were transiently transfected with vectors encoding either wt lck-FLAG or K273M lck-FLAG and seeded into 96-well plates as outlined above. An ELISA was performed with anti-Phospho-Src Family (Tyr416) to detect the amount of phosphorylation of wt lck over the background of K273M lck at the relevant site. Table 2 shows the plate layout of the 2 columns utilized for this analysis and Table 3 shows the absorbance data (raw data) results measured at 450 nM.

TABLE 2
Plate layout for the lck-FLAG ELISA

| | 1 | 2 |
|---|---|---|
| A | wt lck | K273M lck |
| B | wt lck | K273M lck |
| C | wt lck | K273M lck |
| D | wt lck | K273M lck |
| E | K273M lck | wt lck |
| F | K273M lck | wt lck |
| G | K273M lck | wt lck |
| H | K273M lck | wt lck |

TABLE 3
Absorbance results at 450 nM for the lck-FLAG ELISA

| | 1 | 2 |
|---|---|---|
| A | 0.4274 | 0.1106 |
| B | 0.4373 | 0.1048 |
| C | 0.4054 | 0.1026 |
| D | 0.4262 | 0.1002 |
| E | 0.1035 | 0.3928 |
| F | 0.1096 | 0.4146 |
| G | 0.1091 | 0.4181 |
| H | 0.1137 | 0.3791 |

As is seen from the data in Table 3, wt lck-FLAG yielded an average signal of 0.41 in the ELISA, whereas K273M yielded an average signal of 0.11. This indicates that wt lck resulted in approximately 4-fold greater phosphorylation than K273M under these experimental conditions. The reproducibility and Z′ factor for the experiment were good, with standard deviation values of 0.02 for wt lck and 0.005 for K273M lck, and a Z′ factor of 0.76, establishing the suitability of this assay for high-throughput screening applications.

(v.4) Inhibition of lck Phosphorylation with a Known Inhibitory Compound

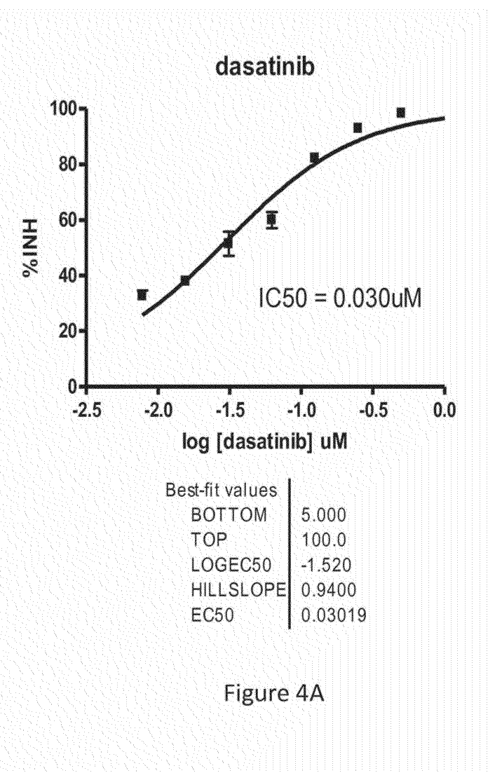

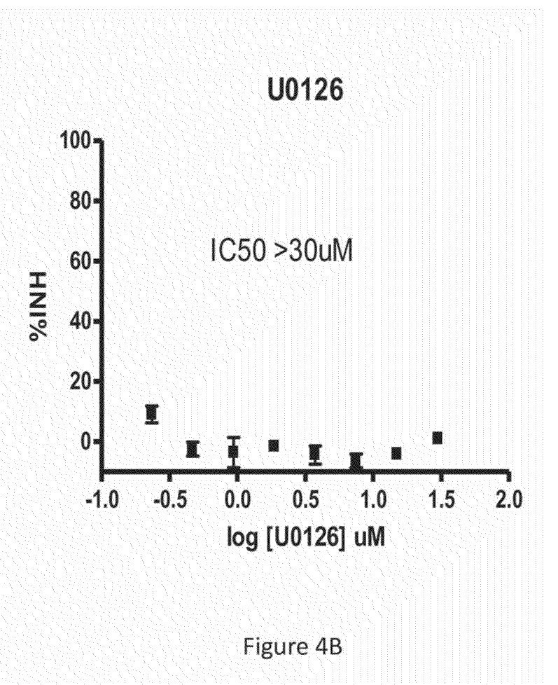

HEK293F cells were transiently transfected and treated with compound for 2 hours, as outlined above, followed by cell lysis and ELISA to detect levels of phosphorylated lck. A dose-response of dasatinib (1, 0.5, 0.25, 0.13, 0.063, 0.031, 0.016, and 0.0078 uM), a known lck inhibitor (obtained from Selleck Chemicals), was used for the compound treatment, as was U0126 (30, 15, 7.5, 3.8, 1.9, 0.94, 0.47, and 0.23 uM), a known MEK inhibitor (EMD Millipore) that should not show inhibitory activity against lck. The raw absorbance data at 450 nM was utilized to calculate percent inhibition values based on their activity relative to uninhibited positive (wt lck-FLAG) and negative (K273M lck-FLAG) controls. Percent inhibition values were plotted vs. the log of compound concentration using GraphPad Prism software with Sigmoidal Dose Response—variable slope curve fitting. The results of this analysis are presented in FIG. 4. FIG. 4 shows the dose response curves for dasatinib, a known lck inhibitor, and U0126, a MEK inhibitor that should not exhibit activity against lck.

Dasatinib inhibited lck in a dose-responsive manner and a 50% inhibitory concentration (IC50) of 30 nM. As expected, the MEK inhibitor U0126 did not inhibit the phosphorylation of lck in this assay (IC50>30 uM).

Taken together, the data in this example indicate that 1) the transiently transfected cells express wt and K273M lck-FLAG, 2) the wt lck-FLAG autophosphorylates at a measurable level above K273M lck, 3) wt lck-FLAG and K273M lck-FLAG can be captured by an anti-FLAG antibody, 4) phosphorylation of lck at its autophosphorylation site can be quantified by ELISA, 5) potencies of compounds against lck can be measured to obtain IC50 values, 6) compounds that do not inhibit lck do not inhibit in the assay, and 7) the assay is suitable for high-throughput screening purposes.

### Example 2

### Pim-2 Ser/Thr Kinase BAD-FLAG Transphosphorylation Assay

Pim (provirus integration site for Moloney murine leukemia virus) genes encode serine/threonine kinases, and are overexpressed in a variety of hematological and epithelial cancers. There are 3 known genes in the Pim family, termed Pim-1, Pim-2, and Pim-3, which are currently being pursued as inhibition targets for the treatment of cancer and other diseases. In this example, FLAG-tagged human BAD is overexpressed in HEK293F cells together with human Pim-2, and Pim-2 phosphorylation of BAD is measured at the equivalent of Ser112 in the mouse sequence. This assay enables potency measurements of Pim-2 kinase inhibitors, as quantitative measurements of Pim-2 dependent BAD phosphorylation can be performed in the presence of differing amounts of inhibitor to generate dose response curves and EC50 values.

(i) Expression Vectors and Antibodies

Plasmid DNA encoding untagged human full length Pim-2 (short isoform) in a mammalian expression vector was obtained from Origene. Plasmid DNA encoding human BAD with a C-terminal FLAG (DDK) and myc tag was obtained from Origene. Kinase-deficient Pim-2 (K61M) was made by mutating the lysine at amino acid 61 to methionine by in vitro site-directed mutagenesis, using the QuikChange XL Site Directed Mutagenesis Kit (Agilent Technologies) under the manufacturer's recommendations. Sequencing was performed to verify that the DNA coding sequence was as expected. Mouse monoclonal anti-FLAG antibody, clone M2, was obtained from Sigma Aldrich. Rabbit monoclonal antibody to BAD, phosphorylated at the site corresponding to Ser112 in the mouse protein, was obtained from Cell Signaling Technology (Phospho-BAD (Ser112) Antibody, clone 40A9). Secondary antibodies (Anti-Rabbit IgG, HRP-linked) were also obtained from Cell Signaling Technology. Rabbit monoclonal antibody to total Pim-2, clone D1D2, and rabbit monoclonal antibody to total BAD (clone D24A9) were also obtained from Cell Signaling Technology.

(ii) Transient Transfection of Mammalian Cells

Human embryonic kidney HEK293F cells (293F; Life Technologies) were grown in T75 flasks to a confluency of approximately 70-80% using the manufacturer's recommended medium supplemented with 10% Fetal Bovine Serum. They were then harvested, washed, and split into 6-well plates (1 T75 per 6 wells) using serum-free medium, which is the same growth medium, but lacking serum. The cells were then serum starved for ˜24 hours. 2 ug of total plasmid DNA was then transfected into each well of the 293F cells using Lipofectamine 2000 (Life Technologies) under the manufacturer's recommendations. Wild type (wt) Pim-2+BAD was utilized for positive controls and either K61M Pim-2+BAD or BAD alone for negative controls. ˜20 hours following transfection, cells were harvested and plated at a density of approximately 1×10⁴ cells per well in 96-well plates. Transfections were also performed in the same way utilizing larger culture vessels (eg. T150 flasks), but scaling the amount of transfection and DNA reagents appropriately.

(iii) Cell Incubation and Compound Treatment

Eight twofold serial dilutions were prepared for each compound in 100% DMSO in 96-well polypropylene plates. Compounds were then diluted in water to 20× final assay concentration and 12% DMSO, and were added to the cells in 96-well plates (20-fold dilution in tissue culture medium) for a final concentration of 1× compound and 0.6% DMSO. For control cells with no compound treatment, DMSO alone was added in a similar manner to achieve a final concentration of 0.6%. Following the addition of compound or DMSO controls, cells were incubated at 37° C. for 2 hours.

(iv) ELISA Autophosphorylation Assay

In preparation for the ELISA, 96-well flat bottom assay plates were coated with anti-FLAG antibody for 1 hour at room temperature or overnight at 4 degrees C., then blocked for 1 hr. with 1% BSA in TBST (TBST consists of 10 mM Tris, pH7.4, 150 mM NaCl and 0.1% Tween 20). Transiently transfected cells treated with compound plus untreated controls in 96-well plates were lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates were then transferred to the coated, blocked ELISA plate. Following 1 hour incubation with lysate, plates were washed 3× with TBST followed by incubation with Phospho-BAD (ser112) primary antibody at 1:1000 dilution in TBST. Following 1 hour incubation with primary antibody, plates were washed 3× with TBST and incubated with a 1:2500 dilution of secondary antibody, HRP-linked anti-rabbit IgG, in TBST. Following 1 hour incubation, plates were washed 3× with TBST, and 1-Step Slow TMB-ELISA reagent (Thermo Scientific) was added. After 10 minute incubation with substrate, equal volume of 2M H₂SO₄ was added, and absorbance was read at 450 nm.

(v) Assay Validation

The Pim-2/BAD-FLAG phosphorylation assay was validated in the following way:

1) verification of wt Pim-2, K61M Pim-2, and BAD expression in transiently transfected 293F cells, detected by Western blotting, 2) verification of BAD phosphorylation when expressed with wt Pim-2, and a reduced amount of BAD phosphorylation when expressed with K61M Pim-2, or expressed alone, in transiently transfected 293F cells following IP with an anti-FLAG antibody and Western blotting, 3) signal for BAD+wt Pim-2 over background of BAD+K61M Pim-2 in the ELISA assay using transiently transfected 293F cells and Phospho-BAD Ser112 as the detection antibody, and 4) a dose dependent reduction in signal for phosphorylated BAD in the ELISA assay using transiently transfected 293F cells and Phospho-BAD Ser112 as the detection antibody, after treatment with a compound that is known to inhibit Pim-2 kinase activity.

(v.1) Verification of Pim-2, K61M Pim-2, and BAD Expression in Transiently Transfected 293F Cells

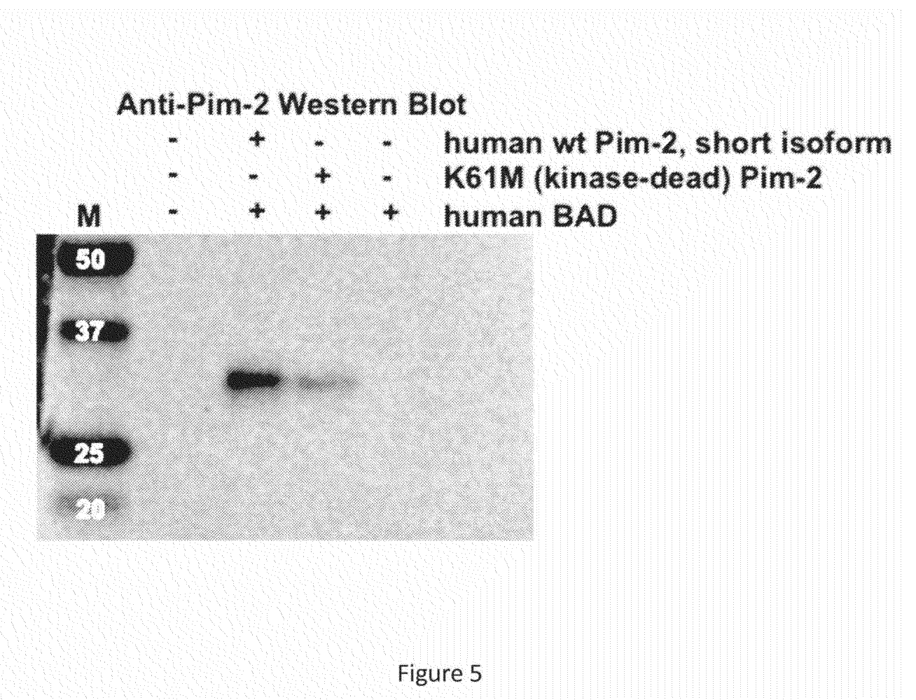

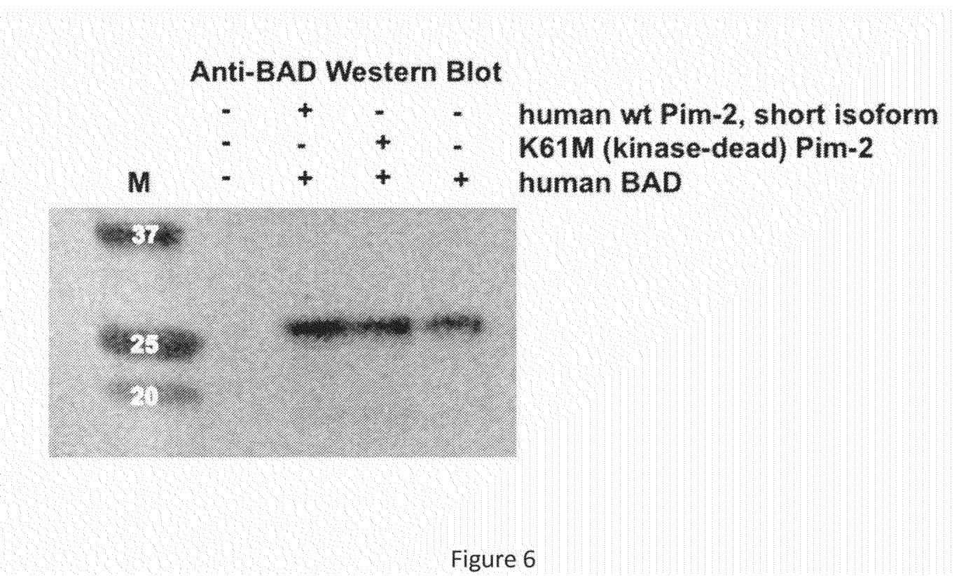

293F cells were transiently transfected in 6-well plates as outlined above, with 1) a vector expressing full length wild type human Pim-2 plus a vector encoding human BAD-FLAG, 2) a vector encoding K61M kinase-deficient mutant full length human Pim-2 plus a vector encoding human BAD-FLAG, and 3) a vector encoding human BAD-FLAG. ˜20 hours after transfection, cells were harvested and lysed in 50 uL 1× lysis buffer per well. 50 uL of 2× gel sample buffer (Bio-Rad) was added and samples were boiled at 95 degrees C. for 3-5 minutes. 10 uL of each sample were run on an Any KD precast polyacrylamide gel (Bio-Rad), followed by transfer to nitrocellulose membrane. Membranes were blocked with 3% BSA in TBST for 1 hour, then probed with 1:1000 anti-(total) Pim-2 antibody or 1:1000 anti-(total) BAD at 4 degrees C. overnight. Following washing 3× with TBST, membranes were probed with HRP-linked anti-rabbit IgG for 1 hour at room temperature. Following washing 3× with TBST, membranes were exposed to the chromogenic HRP Western blotting substrate mixture, CN/DAB (CN/DAB Substrate Kit; Thermo Scientific). Following development of the precipitate, membranes were photographed. The results of such analysis are shown in FIG. 5 (for Pim-2 expression) and FIG. 6 (for BAD expression).

FIG. 5 shows the expression of Pim-2 and K61M Pim-2 in transiently transfected 293F cells. Cells were transfected with the indicated constructs, followed by Western Blotting with anti-Pim-2. Mock transfected cells (no DNA) were used as controls. FIG. 6 shows the expression of BAD-FLAG in transiently transfected 293F cells. Cells were transfected with the indicated constructs, followed by Western Blotting with anti-(total) BAD. Mock transfected cells (no DNA) were used as controls.

These data establish that wt Pim-2, K61M Pim-2, and BAD are all overexpressed in HEK293F cells following transient transfection.

(v.2) Verification of BAD Phosphorylation with Wt Pim-2 Over Background of K61M Pim-2 and BAD Alone in Transiently Transfected 293F Cells

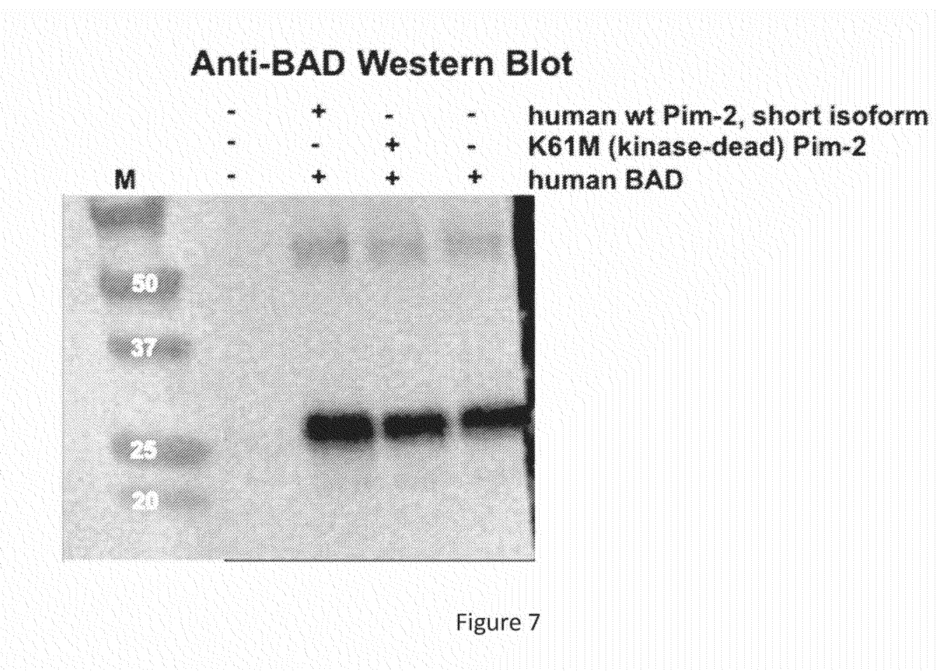

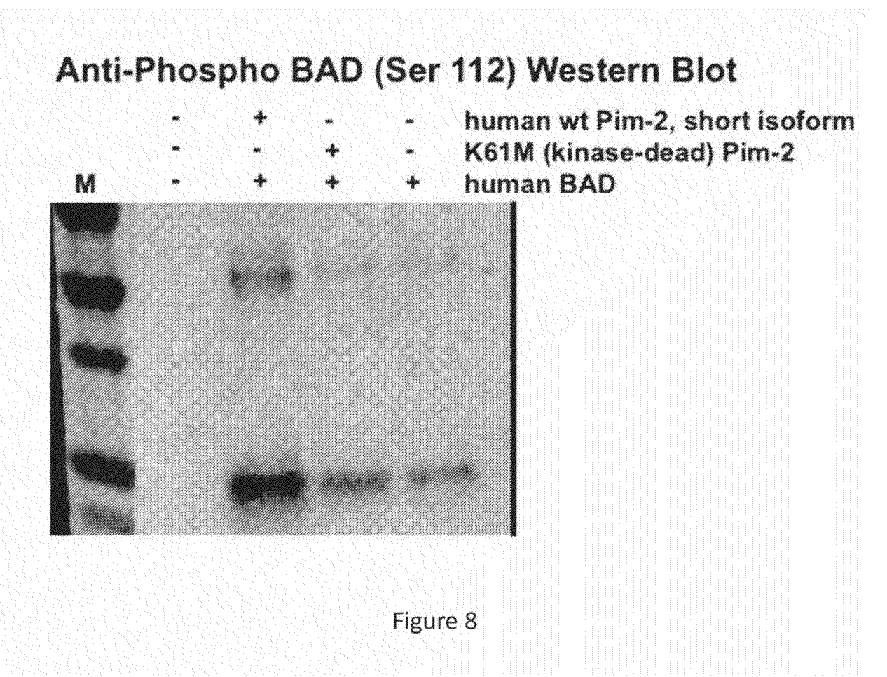

293F cells were transiently transfected in 6-well plates as outlined above, with 1) a vector expressing full length wild type human Pim-2 plus a vector encoding human BAD-FLAG, 2) a vector encoding K61M kinase-deficient mutant full length human Pim-2 plus a vector encoding human BAD-FLAG, and 3) a vector encoding human BAD-FLAG. ˜20 hours after transfection, cells were harvested and lysed in 500 uL 1× lysis buffer per well. Anti-FLAG M2 Affinity Gel (Sigma Aldrich) was used to immunoprecipitate FLAG-tagged BAD under the manufacturer's recommendations. Following IP, 100 uL of 2× gel sample buffer (Bio-Rad) was added and samples were boiled at 95 degrees C. for 3-5 minutes. 10 uL of each sample were run on an Any KD precast polyacrylamide gel (Bio-Rad), followed by transfer to nitrocellulose membrane. Membranes were blocked with 3% BSA in TBST for 1 hour, then probed with either 1:1000 anti-Phospho BAD (Ser112) antibody or 1:1000 anti-total BAD at 4 degrees C. overnight. Following washing 3× with TBST, membranes were probed with HRP-linked anti-rabbit IgG for 1 hour at room temperature. Following washing 3× with TBST, membranes were exposed to the chromogenic HRP Western blotting substrate mixture, CN/DAB (CN/DAB Substrate Kit; Thermo Scientific). Following development of the precipitate, membranes were photographed. The results of such analysis are shown in FIG. 7 and FIG. 8.

FIG. 7 shows the expression of BAD-FLAG in transiently transfected 293F cells. Cells were transfected with the indicated constructs, followed by IP with anti-FLAG and Western Blotting with anti-(total) BAD. Mock transfected cells (no DNA) were used as controls. FIG. 8 shows the phosphorylation of BAD-FLAG in transiently transfected 293F cells. Cells were transfected with the indicated constructs, followed by IP with anti-FLAG and Western Blotting with anti-Phospho BAD (Ser112). Mock transfected cells (no DNA) were used as controls.

Taken together, FIG. 7 and FIG. 8 establish that BAD-FLAG can be immunoprecipitated with anti-FLAG antibody, and that the phosphorylation level of BAD (at the mouse equivalent to Ser112) is higher in cells expressing wt Pim-2 compared to that in cells expressing K61M Pim-2 or BAD alone.

(v.3) ELISA Signal Over Background Measuring BAD Phosphorylation by Pim-2

Cells were transiently transfected with vectors encoding either wt Pim-2 plus BAD-FLAG, or K61M Pim-2 plus BAD-FLAG and seeded into 96-well plates as outlined above. An ELISA was performed with anti-Phospho-BAD (Ser112) to detect the amount of phosphorylation of BAD expressed with Pim-2 over the background of BAD expressed with K61M Pim-2. Table 4 shows the plate layout of the 2 columns utilized for this analysis and Table 5 shows the absorbance data (raw data) results measured at 450 nM following the ELISA.

TABLE 4
Plate layout for the Pim-2/BAD-FLAG phospho-ELISA

| | 1 | 2 |
|---|---|---|
| A | P2 + BAD | K61M P2 + BAD |
| B | P2 + BAD | K61M P2 + BAD |
| C | P2 + BAD | K61M P2 + BAD |
| D | P2 + BAD | K61M P2 + BAD |
| E | K61M P2 + BAD | P2 + BAD |
| F | K61M P2 + BAD | P2 + BAD |
| G | K61M P2 + BAD | P2 + BAD |
| H | K61M P2 + BAD | P2 + BAD |

TABLE 5
Absorbance results at 450 nM for
the Pim-2/BAD-FLAG phospho-ELISA

| | 1 | 2 |
|---|---|---|
| A | 0.628 | 0.179 |
| B | 0.591 | 0.168 |
| C | 0.644 | 0.170 |
| D | 0.684 | 0.176 |
| E | 0.202 | 0.590 |
| F | 0.241 | 0.618 |
| G | 0.175 | 0.604 |
| H | 0.206 | 0.596 |

Table 4 shows the plate layout and Table 5 shows the absorbance results at 450 nM for the Pim-2/BAD-FLAG phospho-ELISA. P2+BAD indicates wells where wt Pim-2 was co-transfected with BAD-FLAG, and K61M P2+BAD indicates wells where kinase-dead K61M Pim-2 was co-transfected with BAD-FLAG.

As is seen from the data in Table 5, BAD-FLAG+wt Pim-2 yielded an average signal of approximately 0.6 in the ELISA, whereas BAD-FLAG+K61M Pim-2 yielded an average signal of about 0.2. This indicates that Pim-2 resulted in approximately 3-fold greater phosphorylation than did K61M toward BAD-FLAG under these experimental conditions. The reproducibility and Z′ factor for the experiment were good, with standard deviation values of ˜0.03 for both wt and K61M Pim-2+BAD, and a Z′ factor of 0.6, establishing the suitability of this assay for high-throughput screening applications.

(v.4) Inhibition of BAD-FLAG Phosphorylation with a Known Inhibitory Compound of Pim-2

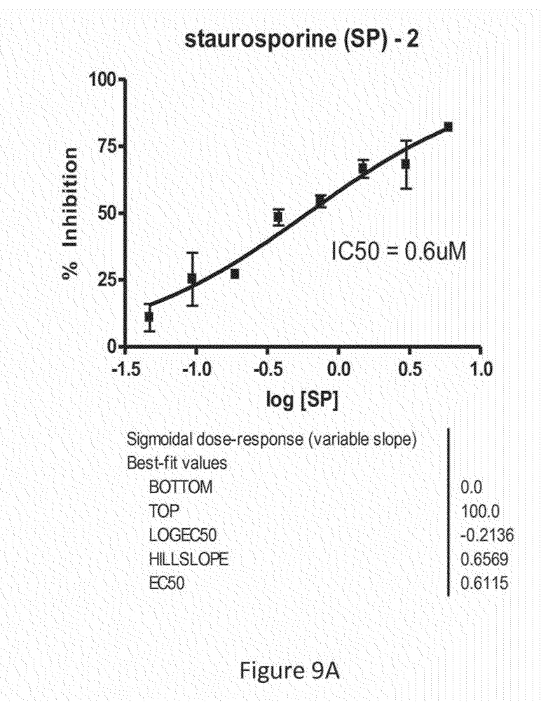

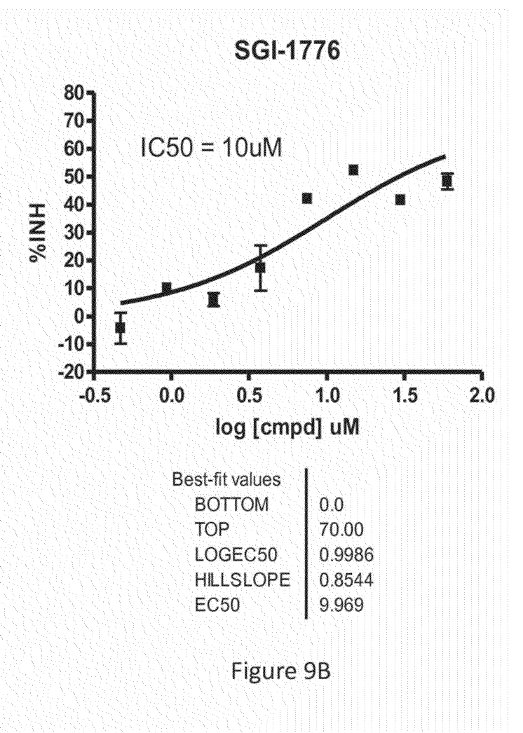

HEK293F cells were transiently transfected and treated with compound for 2 hours, as outlined above, followed by cell lysis and ELISA to detect levels of phosphorylated BAD-FLAG. A dose-response of staurosporine (6, 3, 1.5, 0.75, 0.38, 0.19, 0.094, and 0.047 uM), an inhibitor known to act upon a large number of kinases (obtained from Sigma Aldrich), was used for the compound treatment, as was SGI-1776 (60, 30, 15, 7.5, 3.8, 1.9, 0.94, and 0.47 uM), a known Pim inhibitor (Selleck Chemicals). The raw absorbance data at 450 nM was utilized to calculate percent inhibition values based on their activity relative to uninhibited positive (wt Pim-2+BAD-FLAG) and negative (K61M Pim-2+BAD-FLAG) controls. Percent inhibition values were plotted vs. the log of compound concentration using GraphPad Prism software with Sigmoidal Dose Response—variable slope curve fitting. The results of this analysis are presented in FIGS. 9A and 9B. FIG. 9A shows dose response curve for staurosporine, a general kinase inhibitor, and FIG. 9B shows dose response curve for SGI-1776, a more specific inhibitor that should exhibit activity against Pim-2.

Staurosporine inhibited Pim-2 in a dose-responsive manner and a 50% inhibitory concentration (IC50) of approximately 600 nM. As expected, the Pim inhibitor SGI-1776 also inhibited the phosphorylation of BAD in this assay (IC50 of approximately 10 uM).

Taken together, the data in this example indicate that 1) the transiently transfected cells express wt Pim-2, K61M Pim-2, and BAD-FLAG, 2) the wt Pim-2 phosphorylates BAD at a measurable level above that measured with K61M Pim-2, 3) BAD-FLAG can be captured by an anti-FLAG antibody, 4) phosphorylation of BAD at the site equivalent to Ser112 in mouse can be quantified by ELISA, 5) potencies of compounds against Pim-2 can be measured to obtain IC50 values, and 6) the assay is suitable for high-throughput screening purposes.

### Example 3

### FGFR1-Myc and EphB4-FLAG Multiplexed Tyrosine Kinase Autophosphorylation Assay

This example is presented to indicate the utility of the described assay system for testing more than 1 tyrosine kinase for inhibition with compounds within the same cells. This will utilize 2 different receptor tyrosine kinases, namely FGFR1 and EphB4, and 2 different affinity tags, namely myc and FLAG.

(i) Expression Vectors and Antibodies

Plasmid DNA encoding human full length FGFR1 with a C-terminal myc tag, in a mammalian expression vector, will be obtained commercially or synthesized, as will a mammalian expression vector expressing full length human EphB4 with a C-terminal FLAG tag. Kinase-deficient mutants will be made by mutating the relevant sites by in vitro site-directed mutagenesis, using the QuikChange XL Site Directed Mutagenesis Kit (Agilent Technologies) under the manufacturer's recommendations. Sequencing will be performed to verify that the DNA coding sequence is as expected. Mouse monoclonal anti-FLAG antibody, clone M2, will be obtained from Sigma Aldrich. Mouse monoclonal anti-myc antibody will be obtained from Cell Signaling Technology. Monoclonal antibody to antiphosphotyrosine, directly linked to HRP, will be obtained from a commercial source. Polyclonal or monoclonal antibodies to total FGFR1 and total EphB4 will also be obtained from a commercial source.

(ii) Transient Transfection of Mammalian Cells

Human embryonic kidney HEK293F cells (293F; Life Technologies) will be seeded in 6-well plates and grown to a confluency of approximately 70-80% using the manufacturer's recommended medium supplemented with 10% Fetal Bovine Serum. 1 ug of plasmid DNA encoding human full length FGFR1-myc and 1 ug of plasmid encoding human full length EphB4-FLAG will be co-transfected into each well of the 293F cells using Lipofectamine 2000 (Life Technologies) under the manufacturer's recommendations. Plasmids encoding corresponding kinase-deficient mutants will also be utilized to transfect cells in the same manner, for negative controls. ˜20 hours following transfection, cells will be harvested and plated at a density of approximately 2×10⁴ cells per well in 96-well plates. Transfections will also be performed in the same way utilizing larger culture vessels (eg. T75 flasks), but scaling the amount of transfection and DNA reagents appropriately.

(iii) Cell Incubation and Compound Treatment

Eight twofold serial dilutions will be prepared for each compound in 100% DMSO in 96-well polypropylene plates. Compounds will then be diluted in water to 20× final assay concentration and 12% DMSO, and added to the cells in 96-well plates (20-fold dilution in tissue culture medium) for a final concentration of 1× compound and 0.6% DMSO. For control cells with no compound treatment, DMSO alone will be added in a similar manner to achieve a final concentration of 0.6%. Following the addition of compound or DMSO controls, cells will be incubated at 37° C. for 2 hours.

(iv) ELISA Autophosphorylation Assay

In preparation for the ELISA, 96-well flat bottom assay plates will be coated with either anti-FLAG antibody or anti-myc antibody for 1 hour at room temperature or overnight at 4 degrees C., then blocked for 1 hr. with 1% BSA in TBST (TBST consists of 10 mM Tris, pH7.4, 150 mM NaCl and 0.1% Tween 20). Transiently transfected cells treated with compound plus untreated controls in 96-well plates will be lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates from each plate will then be split and transferred to both coated, blocked ELISA plates. Following 1 hour incubation with lysate, plates will be washed 3× with TBST followed by incubation with HRP-linked antiphosphotyrosine antibody at 1:5000 dilution in TBST. Following 1 hour incubation with antibody, plates will be washed 3× with TBST, and 1-Step Slow TMB-ELISA reagent (Thermo Scientific) will be added. After 10 minute incubation with substrate, equal volume of 2M H₂SO₄ will be added, and absorbance will be read at 450 nm.

(v) Assay Validation and Expected Results

The FGFR1-myc/EphB4-FLAG multiplexed autophosphorylation assay will be validated in the following way: 1) verification of wt FGFR1, kinase dead FGFR1, wt EphB4, and kinase dead EphB4 expression in transiently transfected 293F cells, following immunoprecipitation (IP) with anti-FLAG and anti-myc antibodies and Western blotting, 2) verification of wt FGFR1 and wt EphB4 autophosphorylation, and much reduced levels of phosphorylation with corresponding kinase dead mutants, in transiently transfected 293F cells following IP with anti-FLAG and anti-myc antibodies and Western blotting, 3) signal for wt FGFR1 and wt EphB4 over background of corresponding kinase deficient mutants in the ELISA assay with matched capture antibodies for the relevant affinity tags and using transiently transfected 293F cells and antiphosphotyrosine as the detection antibody, 4) lack of cross-reactivity between anti-FLAG and anti-myc capture antibodies and FLAG and myc affinity tags, 5) a dose dependent reduction in signal for phosphorylated wt FGFR1 and wt EphB4 in the ELISA assay using transiently transfected 293F cells and antiphosphotyrosine as the detection antibody, after treatment with a compound that is known to inhibit FGFR1 and/or EphB4 kinase activity, but not with a compound that does not inhibit these kinases, and 6) similar IC50 values for compound treatment with both kinases in the multiplex format as are obtained when performing the assay for each kinase alone (singleplex format).

Taken together, the data in this prophetic example are expected to indicate that 1) the transiently transfected cells express wt FGFR1, kinase dead FGFR1, wt EphB4, and kinase dead EphB4, 2) the wt FGFR1 autophosphorylates at a higher level than kinase dead FGFR1 and wt EphB4 autophosphorylates at a higher level than kinase dead EphB4, 3) FGFR1-myc can be captured by the anti-myc antibody, EphB4-FLAG can be captured by an anti-FLAG antibody, and that there is no cross-reactivity between anti-myc and FLAG or anti-FLAG and myc, or between the antibodies and kinases, 4) autophosphorylation of the kinases over kinase deficient mutants can be quantified by ELISA, 5) potencies of compounds against FGFR1 and EphB4 can be measured together to obtain IC50 values that match values obtained with the treatment of either alone, and 6) that the assay is suitable for high-throughput screening purposes.

### Example 4

### lck-HA and Pim2/BAD-FLAG Multiplexed Kinase Assay

This example is presented to indicate the utility of the described assay system for testing a serine/threonine and a tyrosine kinase together for inhibition with compounds within the same cells. This will utilize lck as the tyrosine kinase and Pim-2/BAD as the serine/threonine kinase, substrate pair. HA and FLAG will be utilized as the 2 different affinity tags.

(i) Expression Vectors and Antibodies

Plasmid DNA encoding human full length lck with a C-terminal HA tag, in a mammalian expression vector, will be obtained commercially or synthesized, as will mammalian expression vectors expressing full length human Pim-2 (untagged) and human BAD with a C-terminal FLAG tag. Kinase-deficient mutants of lck and Pim-2 will be made by mutating the relevant sites by in vitro site-directed mutagenesis, using the QuikChange XL Site Directed Mutagenesis Kit (Agilent Technologies) under the manufacturer's recommendations. Sequencing will be performed to verify that the DNA coding sequence is as expected. Mouse monoclonal anti-FLAG antibody, clone M2, will be obtained from Sigma Aldrich. Mouse monoclonal anti-HA antibody will be obtained from Sigma Aldrich. Polyclonal antibody to the autophosphorylation site in lck, which is equivalent to the Y416 site in the human family member src, will be obtained from Cell Signaling Technology (Phospho-Src Family (Tyr416) Antibody). Secondary antibodies (Anti-Rabbit IgG, HRP-linked) will also be obtained from Cell Signaling Technology. Polyclonal antibody to the N-terminus of lck will be obtained from Abgent. Rabbit monoclonal antibody to BAD, phosphorylated at the site corresponding to Ser112 in the mouse protein, will be obtained from Cell Signaling Technology (Phospho-BAD (Ser112) Antibody, clone 40A9). Rabbit monoclonal antibody to total Pim-2, clone D1D2, and rabbit monoclonal antibody to total BAD (clone D24A9) will also be obtained from Cell Signaling Technology.

(ii) Transient Transfection of Mammalian Cells

Human embryonic kidney HEK293F cells (293F; Life Technologies) will be grown in T75 flasks to a confluency of approximately 70-80% using the manufacturer's recommended medium supplemented with 10% Fetal Bovine Serum. They will then be harvested, washed, and split into 6-well plates (1 T75 per 6 wells) using serum-free medium, which is the same growth medium, but lacking serum. The cells will then be serum starved for ˜24 hours. 0.67 ug of plasmid DNA encoding human full length lck-HA, 0.67 ug of plasmid DNA encoding human full length Pim-2, and 0.67 ug of plasmid encoding human full length BAD-FLAG will be co-transfected into each well of the 293F cells using Lipofectamine 2000 (Life Technologies) under the manufacturer's recommendations. Plasmids encoding corresponding kinase-deficient mutants will also be utilized to transfect cells in the same manner, for negative controls. ˜20 hours following transfection, cells will be harvested and plated at a density of approximately 2-4×10⁴ cells per well in 96-well plates. Transfections will also be performed in the same way utilizing larger culture vessels (eg. T75 flasks), but scaling the amount of transfection and DNA reagents appropriately.

(iii) Cell Incubation and Compound Treatment

Eight twofold serial dilutions will be prepared for each compound in 100% DMSO in 96-well polypropylene plates. Compounds will then be diluted in water to 20× final assay concentration and 12% DMSO, and added to the cells in 96-well plates (20-fold dilution in tissue culture medium) for a final concentration of 1× compound and 0.6% DMSO. For control cells with no compound treatment, DMSO alone will be added in a similar manner to achieve a final concentration of 0.6%. Following the addition of compound or DMSO controls, cells will be incubated at 37° C. for 2 hours.

(iv) ELISA Autophosphorylation Assay

In preparation for the ELISA, 96-well flat bottom assay plates will be coated with either anti-FLAG antibody or anti-HA antibody for 1 hour at room temperature or overnight at 4 degrees C., then blocked for 1 hr. with 1% BSA in TBST (TBST consists of 10 mM Tris, pH7.4, 150 mM NaCl and 0.1% Tween 20). Transiently transfected cells treated with compound plus untreated controls in 96-well plates will be lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates from each plate will then be split and transferred to both coated, blocked ELISA plates. Following 1 hour incubation with lysate, plates will be washed 3× with TBST followed by incubation with either anti-Phospho-Src Family (Tyr416) antibody at 1:1000 dilution in TBST for the anti HA-coated plate or anti-Phospho-BAD (Ser112) at 1:1000 dilution in TBST for the anti FLAG-coated plate. Following 1 hour incubation with primary antibody, plates will be washed 3× with TBST and incubated with a 1:2500 dilution of secondary antibody, HRP-linked anti-rabbit IgG, in TBST. Following 1 hour incubation, plates will be washed 3× with TBST, and 1-Step Slow TMB-ELISA reagent (Thermo Scientific) will be added. After 10 minute incubation with substrate, equal volume of 2M H₂SO₄ will be added, and absorbance will be read at 450 nm.

(v) Assay Validation and Expected Results

The lck-HA+Pim-2/BAD-FLAG multiplexed kinase assay will be validated in the following way: 1) verification of wt lck, kinase dead lck, wt Pim-2, kinase dead Pim-2, and BAD expression in transiently transfected 293F cells following Western blotting, 2) verification of wt lck and BAD phosphorylation with Pim-2 expression, and much reduced levels of phosphorylation with corresponding kinase dead mutants (kinase dead lck autophosphorylation and BAD phosphorylation with kinase dead Pim-2), in transiently transfected 293F cells following IP with anti-FLAG and anti-HA antibodies and Western blotting, 3) signal for wt lck and BAD (expressed with wt Pim-2) over background of corresponding kinase deficient mutants (kinase dead lck autophosphorylation and BAD phosphorylation with expression of kinase dead Pim-2) in the ELISA assay with matched capture antibodies for the relevant affinity tags and using transiently transfected 293F cells and phospho-specific detection antibodies, 4) lack of cross-reactivity between anti-FLAG and anti-HA capture antibodies and FLAG and HA affinity tags, 5) a dose dependent reduction in signal for phosphorylated wt lck and BAD expressed with wt Pim-2 in the ELISA assay using transiently transfected 293F cells and relevant phospho-specific antibodies for detection, after treatment with a compound that is known to inhibit lck and/or Pim-2 kinase activity, but not with a compound that does not inhibit these kinases, and 6) similar IC50 values for compound treatment with both kinases in the multiplex format as are obtained when performing the assay for each kinase alone (singleplex format).

Taken together, the data in this prophetic example are expected to indicate that 1) the transiently transfected cells express wt lck, kinase dead lck, wt Pim-2, kinase dead Pim-2, and BAD, 2) the wt lck autophosphorylates at a higher level than kinase dead lck and wt Pim-2 results in a greater phosphorylation level of BAD than kinase dead Pim-2, 3) lck-HA can be captured by the anti-HA antibody, BAD-FLAG can be captured by an anti-FLAG antibody, and that there is no cross-reactivity between anti-HA and FLAG or anti-FLAG and HA, or between the antibodies and kinases, or substrate, 4) autophosphorylation and phosphorylation of the substrate above the levels seen with kinase deficient mutants can be quantified by ELISA, 5) potencies of compounds against lck and Pim-2 can be measured together to obtain IC50 values that match values obtained with the treatment of either alone, and 6) the assay is suitable for high-throughput screening purposes.

### Example 5

### DOT1L Methyltransferase Histone H3-FLAG Methylation Assay

This example is intended to show the utility of the described assay system for measuring methylation as a post-translational modification. The Histone H3 Lysine 79 Methyltransferase DOT1L/KMT4 will be overexpressed in human cells along with a FLAG-tagged histone H3 as the substrate. Methylation at site 79 on the substrate will be measured following cell lysis and capture of the histone H3 with anti-FLAG antibody in an ELISA assay.

(i) Expression Vectors and Antibodies

Plasmid DNA encoding untagged human full length human DOT1L in a mammalian expression vector will be obtained commercially or synthesized, along with plasmid DNA encoding human histone H3 with a FLAG tag. Methylation-deficient DOT1L will be made by mutating the relevant site in the active site by in vitro site-directed mutagenesis, using the QuikChange XL Site Directed Mutagenesis Kit (Agilent Technologies) under the manufacturer's recommendations. Sequencing will be performed to verify that the DNA coding sequence is as expected. Mouse monoclonal anti-FLAG antibody, clone M2, will be obtained from Sigma Aldrich. Antibody to the methylated lysine at amino acid 79 on histone H3 will be obtained commercially (eg. from Cell Signaling Technology). Secondary antibodies (Anti-Rabbit IgG, HRP-linked or Anti-Mouse IgG, HRP-linked) will also be obtained from Cell Signaling Technology. Antibodies to total DOT1L and total histone H3 will also be obtained commercially.

(ii) Transient Transfection of Mammalian Cells

Human cells will be grown in T75 flasks to a confluency of approximately 70-80% using the manufacturer's recommended medium supplemented with 10% Fetal Bovine Serum. They will then be harvested, washed, and split into 6-well plates (1 T75 per 6 wells) using serum-free medium, which is the same growth medium, but lacking serum. The cells will then be serum starved for ˜24 hours. 2 ug of total plasmid DNA will be transfected into each well of the cells using Lipofectamine 2000 (Life Technologies) under the manufacturer's recommendations. Vectors encoding wild type (wt) DOT1L plus histone H3-FLAG will be utilized for positive controls and vectors encoding either methyltransferase deficient DOT1L+histone H3-FLAG or histone H3-FLAG alone for negative controls. ˜20 hours following transfection, cells will be harvested and plated at a density of approximately 1×10⁴ cells per well in 96-well plates. Transfections will also be performed in the same way utilizing larger culture vessels (eg. T150 flasks), but scaling the amount of transfection and DNA reagents appropriately.

(iii) Cell Incubation and Compound Treatment

Eight twofold serial dilutions will be prepared for each compound in 100% DMSO in 96-well polypropylene plates. Compounds will then be diluted in water to 20× final assay concentration and 12% DMSO, and will be added to the cells in 96-well plates (20-fold dilution in tissue culture medium) for a final concentration of 1× compound and 0.6% DMSO. For control cells with no compound treatment, DMSO alone will be added in a similar manner to achieve a final concentration of 0.6%. Following the addition of compound or DMSO controls, cells will be incubated at 37° C. for a period of 3-14 days, with replenishment of the medium as necessary.

(iv) ELISA Methylation Assay

In preparation for the ELISA, 96-well flat bottom assay plates will be coated with anti-FLAG antibody for 1 hour at room temperature or overnight at 4 degrees C., then blocked for 1 hr. with 1% BSA in TBST (TBST consists of 10 mM Tris, pH7.4, 150 mM NaCl and 0.1% Tween 20). Transiently transfected cells treated with compound plus untreated controls in 96-well plates will be lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates will then be transferred to the coated, blocked ELISA plate. Following 1 hour incubation with lysate, plates will be washed 3× with TBST followed by incubation with anti-methyl lysine 79 antibody diluted in TBST. Following 1 hour incubation with primary antibody, plates will be washed 3× with TBST and incubated with a 1:2500 dilution of secondary antibody in TBST. Following 1 hour incubation, plates will be washed 3× with TBST, and 1-Step Slow TMB-ELISA reagent (Thermo Scientific) will be added. After 10 minute incubation with substrate, equal volume of 2M H₂SO₄ will be added, and absorbance will be read at 450 nm.
(v) Assay Validation

The DOT1L/Histone H3-FLAG methylation assay will be validated in the following way: 1) verification of wt DOT1L, methyltransferase deficient DOT1L, and histone H3 expression in transiently transfected cells, as detected by Western blotting, 2) verification of histone H3 methylation when expressed with wt DOT1L, and a reduced amount of histone H3 methylation when expressed with methyltransferase deficient DOT1L, or expressed alone, in transiently transfected cells following IP with an anti-FLAG antibody and Western blotting, 3) signal for histone H3+wt DOT1L over background of histone H3+methyltransferase deficient DOT1L in the ELISA assay using transiently transfected cells and histone H3 methyl lysine 79 specific antibody as the detection antibody, and 4) a dose dependent reduction in signal for methylated histone H3 in the ELISA assay using transiently transfected cells and methyl lysine 79 specific antibody as the detection antibody, after treatment with a compound that is known to inhibit DOT1L methyltransferase activity.

Taken together, the data in this prophetic example are expected to indicate that 1) the transiently transfected cells express wt DOT1L, methyltransferase deficient DOT1L, and histone H3-FLAG, 2) the wt DOT1L results in higher levels of histone H3 methylation at Lys 79 compared to levels with methyltransferase deficient DOT1L, 3) Histone H3-FLAG can be captured by an anti-FLAG antibody, 4) methylation dependent on DOT1L methyltransferase activity can be quantified by ELISA, 5) potencies of compounds against DOT1L can be measured to obtain IC50 values, and 6) the assay is suitable for high-throughput screening purposes.

### Example 6

### FGFR1-Myc and Abl-FLAG Multiplexed Tyrosine Kinase Autophosphorylation

This example is presented to indicate the utility of the described system for testing more than 1 tyrosine kinase for autophosphorylation within the same cells. The example utilizes 2 different receptor tyrosine kinases, namely FGFR1 and Abl, and 2 different affinity tags, namely myc and FLAG.

(i) Expression Vectors and Antibodies

Plasmid DNA encoding human full length FGFR1, transcript variant 1, with a C-terminal FLAG (DDK) and myc tag, in a mammalian expression vector, was obtained from Origene. The FLAG tag portion of the expression vector was modified by performing site-directed mutagenesis to create a stop codon causing premature termination before the FLAG tag, thus resulting in FGFR1 expression with only a myc tag. Plasmid DNA encoding full length human c-Abl 1, transcript variant a, with a C-terminal FLAG tag, was constructed by performing DNA synthesis utilizing the Abl DNA sequence (NM_{—}005157.3) and inserting it into a pCMV6-based vector engineered to express proteins with a C-terminal FLAG-tag. Kinase-deficient mutants were made by mutating the relevant sites by in vitro site-directed mutagenesis (FGFR1 [K512M] and Abl [K213M]), using standard techniques. Sequencing was performed to verify that the DNA coding sequence is as expected. Mouse monoclonal anti-FLAG antibody, clone M2, was obtained from Sigma Aldrich. Mouse monoclonal anti-myc antibody was obtained from Cell Signaling Technology. Monoclonal antibody to antiphosphotyrosine, directly linked to HRP, was obtained from Santa Cruz Biotechnology.

(ii) Transient Transfection of Mammalian Cells

Human embryonic kidney HEK293F cells (293F; Life Technologies) were seeded in 6-well plates and grown to a confluency of approximately 70-80% using the manufacturer's recommended medium supplemented with 10% Fetal Bovine Serum. 1.5 ug of plasmid DNA encoding human full length FGFR1-myc and 1.5 ug of plasmid encoding human full length Abl-FLAG were co-transfected into each well of the 293F cells using Lipofectamine 2000 (Life Technologies) under the manufacturer's recommendations. Plasmids encoding corresponding kinase-deficient mutants were also utilized to transfect cells in the same manner, for negative controls. ˜20 hours following transfection, cells were harvested and plated at a density of approximately 3×10⁴ cells per well in 96-well plates.

(iii) ELISA Multiplex Autophosphorylation Assay

In preparation for the ELISA, 8 wells of a 96-well flat bottom assay plate were coated with 1 ug each of anti-FLAG antibody and 8 wells were coated with 1 ug each of anti-myc antibody for 1 hour at room temperature, then blocked for 1 hr. with 1% BSA in TBST (TBST consists of 10 mM Tris, pH7.4, 150 mM NaCl and 0.1% Tween 20). Transiently transfected cells that were co-transfected with FGFR1-myc and Abl-FLAG and transiently transfected controls co-transfected with kinase-dead FGFR1-myc and kinase-dead Abl-FLAG, obtained as above, were lysed by the addition of 5× lysis buffer (5× lysis buffer consists of 100 mM Tris, pH7.5, 750 mM NaCl, 5 mM EDTA, 5 mM EGTA, 5% TritonX-100, 5 mM PMSF plus 5× Halt Protease & Phosphatase inhibitor Cocktail (Thermo Scientific)) to a final concentration of 1×, and incubated at room temperature for 10 minutes with shaking. Lysates were then transferred to the coated, blocked ELISA plate. Following 1 hour incubation with lysate, plates were washed 3× with TBST followed by incubation with HRP-linked PY99 antibody at 1:5000 dilution in TBST. Following 1 hour incubation, plates were washed 3× with TBST, and 1-Step Slow TMB-ELISA reagent (Thermo Scientific) was added. After 10 minute incubation with substrate, equal volume of 2M H₂SO₄ was added, and absorbance was read at 450 nm.
(iv) ELISA Multiplex Results

Cells were transiently co-transfected with vectors encoding either wt FGFR1-myc and wt Abl-FLAG or kinase-deficient FGFR1-myc and kinase-deficient Abl-FLAG and seeded into 96-well plates as detailed above. An ELISA was performed with PY99 following capture with anti-FLAG and, separately, capture with anti-myc, to detect the amount of phosphorylation of wt FGFR1 and wt Abl over the background of kinase deficient mutants, within the same cells. Tables 6 and 7 show the plate layout of the 2 columns utilized for this analysis along with the absorbance data (raw data) results measured at 450 nm.

TABLE 6
Plate layout and Absorbance results for the multiplexed
FGFR1-myc and Abl-FLAG ELISA showing positive controls

| Sample | Coating Ab | Cell Density | Abs 450 nm |
|---|---|---|---|
| wt FGFR1 + wt Abl | anti-FLAG | 30,000 | 0.797 |
| wt FGFR1 + wt Abl | anti-FLAG | 15,000 | 0.692 |
| wt FGFR1 + wt Abl | anti-FLAG | 7,500 | 0.519 |
| wt FGFR1 + wt Abl | anti-FLAG | 3,750 | 0.353 |
| wt FGFR1 + wt Abl | anti-myc | 30,000 | 0.935 |
| wt FGFR1 + wt Abl | anti-myc | 15,000 | 0.939 |
| wt FGFR1 + wt Abl | anti-myc | 7,500 | 0.917 |
| wt FGFR1 + wt Abl | anti-myc | 3,750 | 0.856 |

TABLE 7
Plate layout and Absorbance results at 450 nm for the multiplexed
FGFR1-myc and Abl-FLAG ELISA showing negative controls

| Sample | Coating Ab | Cell Density | Abs 450 nm |
|---|---|---|---|
| kd FGFR1 + kd Abl | anti-FLAG | 30,000 | 0.091 |
| kd FGFR1 + kd Abl | anti-FLAG | 15,000 | 0.090 |
| kd FGFR1 + kd Abl | anti-FLAG | 7,500 | 0.086 |
| kd FGFR1 + kd Abl | anti-FLAG | 3,750 | 0.100 |
| kd FGFR1 + kd Abl | anti-myc | 30,000 | 0.082 |
| kd FGFR1 + kd Abl | anti-myc | 15,000 | 0.111 |
| kd FGFR1 + kd Abl | anti-myc | 7,500 | 0.093 |
| kd FGFR1 + kd Abl | anti-myc | 3,750 | 0.114 |

As is seen in the data from Tables 6 and 7, the ELISA signal for phosphorylation of FGFR1 (using anti-myc capture) and phosphorylation of Abl (using anti-FLAG capture) are several fold higher than the corresponding ELISA signals for the corresponding kinase-dead versions of the kinases. This indicates that FGFR1 and Abl were both phosphorylated following co-transfection in human cells, and that multiplexing yielded a signal over control for both kinases. This clearly illustrates the feasibility of multiplexing the activities of more than one tyrosine kinase in this assay system.

Other embodiments and uses will be apparent to those skilled in the art from consideration of the specification and practice of the methods and constructs disclosed herein. All U.S. Patents and other references noted herein for whatever reason are specifically incorporated by reference. The specification and examples should be considered exemplary only with the true scope and spirit of the invention indicated by the following claims.

What is claimed is:

1. A multiplex method for assaying the effect of a test compound on the activity of multiple kinases in a biological cell, the method comprising the steps of:
(a) obtaining a plurality of transiently transfected biological cells that transiently express (i) at least a first target kinase and a second target kinase, and (ii) at least one first specific protein substrate of the first target kinase and at least one second specific protein substrate of the second target kinase, wherein the first specific protein substrate is linked to at least one first capture tag and the second specific protein substrate is linked to at least one second capture tag, and wherein the first and second capture tags are not conjugated to rare earth elements and are not identical;
(b) culturing the plurality of biological cells under conditions suitable to transiently express the first and second target kinases and the first and second specific protein substrates;
(c) incubating the plurality of biological cells, in at least one first multi-well plate, with at least one test compound under conditions suitable to allow phosphorylation of the first and second specific protein substrates by the first and second target kinases;
(d) lysing the plurality of biological cells, transferring the lysed cells to at least one second multi-well plate, and capturing (i) the first specific protein substrate using at least one capture antibody that specifically binds the first capture tag, and (ii) the second specific protein substrate using at least one capture antibody that specifically binds the second capture tag;
(e) detecting the presence of phosphorylated first and second specific protein substrates using (i) at least one first detectable antibody that specifically binds the phosphorylated form of the first specific protein substrate, but does not substantially bind the first specific protein substrate when not phosphorylated, and (ii) at least one second detectable antibody that specifically binds the phosphorylated form of the second specific protein substrate, but does not substantially bind the second specific protein substrate when not phosphorylated; and
(f) comparing the level of phosphorylated first and second specific protein substrates detected in step (e) with 1) the level of phosphorylated first and second specific protein substrates in at least one control sample taken from control biological cells not contacted with the test compound, and 2) the level of phosphorylated first and second specific protein substrates in at least one control sample taken from control biological cells not contacted with the test compound but rather treated with known target kinase inhibitor(s), or otherwise equivalent cells expressing kinase-deficient mutant instead of active forms of the target kinases, thereby assaying the effect of the test compound on the activity of the first and second target kinases within the biological cells of step (c).

2. The multiplex method of claim 1, wherein the first and second specific protein substrates are each not a plurality of promiscuous peptide substrates capable of being phosphorylated by the first and second target kinases or other kinases.

3. The multiplex method of claim 1, wherein the first and second target kinases are tyrosine kinases, wherein the first specific protein substrate is a domain of the first tyrosine kinase that is auto-phosphorylated by the kinase domain of the first tyrosine kinase, and wherein the second specific protein substrate is a domain of the second tyrosine kinase that is auto-phosphorylated by the kinase domain of the second tyrosine kinase.

4. The multiplex method of claim 1, wherein the first and second target kinases are serine/threonine kinases, wherein the first specific protein substrate is (i) a domain of the first serine/threonine kinase that is auto-phosphorylated by the first serine/threonine kinase or (ii) an independent non-domain substrate that is phosphorylated by the first serine/threonine kinase, wherein the second specific protein substrate is (iii) a domain of the second serine/threonine kinase that is auto-phosphorylated by the first serine/threonine kinase or (iv) an independent non-domain substrate that is phosphorylated by the second serine/threonine kinase, and wherein the first and second specific protein substrates are not identical with respect to either sequence, phosphorylation site, epitope tag, or any combination thereof.

5. The multiplex method of claim 4, wherein the biological cells of step (a) have been transiently co-transfected with (i) at least one first polynucleotide comprising a polynucleotide encoding the first serine/threonine kinase, (ii) at least one second polynucleotide comprising a polynucleotide encoding the first specific protein substrate, (iii) at least one third polynucleotide comprising a polynucleotide encoding the second serine/threonine kinase, and optionally (iv) at least one fourth polynucleotide comprising a polynucleotide encoding the second specific protein substrate.

6. The multiplex method of claim 5, wherein the first and second polynucleotides are operably linked to express a first fusion protein comprising the first serine/threonine kinase and the first specific protein substrate, and wherein the third and fourth polynucleotides are operably linked to express a second fusion protein comprising the second serine/threonine kinase and the second specific protein substrate.

7. The multiplex method of claim 1, wherein the first target kinase is a tyrosine kinase, wherein the second target kinase is a serine/threonine kinase, wherein the first specific protein substrate is a domain of the tyrosine kinase that is auto-phosphorylated by the kinase domain of the tyrosine kinase, and wherein the second specific protein substrate is (i) a domain of the serine/threonine kinase that is auto-phosphorylated by serine/threonine kinase or (ii) an independent non-domain substrate that is phosphorylated by the serine/threonine kinase.

8. The multiplex method of claim 7, wherein the biological cells of step (a) have been transiently co-transfected with (i) at least one first polynucleotide comprising a polynucleotide encoding the tyrosine kinase, (ii) at least one second polynucleotide comprising a polynucleotide encoding the serine/threonine kinase, and optionally (iii) at least one third polynucleotide comprising a polynucleotide encoding the specific protein substrate of the serine/threonine kinase.

9. The multiplex method of claim 8, wherein the second and third polynucleotides are operably linked to express a fusion protein comprising the serine/threonine kinase and the specific protein substrate.

10. The multiplex method of claim 5, wherein the biological cells of step (a) have been serum starved prior to being transiently co-transfected.

11. The multiplex method of claim 1, wherein the transiently transfected biological cells comprise an expression vector that comprises first polynucleotide encoding the first target kinase and a second polynucleotide encoding the second target kinase.

12. The multiplex method of claim 5, wherein the first, second, third, and fourth polynucleotides are comprised within a single expression vector.

13. The multiplex method of claim 8, wherein the first, second, and third polynucleotides are comprised within a single expression vector.

14. The multiplex method of claim 1, wherein the transiently transfected biological cells of step (a) comprise human embryonic kidney (HEK) 293 cells capable of expressing the first and second target kinases, and the first and second specific substrates, within about 1-5 days of being transfected, and wherein the transiently transfected HEK 293 cells are not stably transformed and do not stably express the first and second target kinases or the first and second specific protein substrates, except in endogenous form.

15. The multiplex method of claim 1, wherein the first and second capture tags are selected from the group consisting of FLAG, 3×FLAG, Myc, HA, HIS, 3×HIS, Isopeptag, BCCP, Calmodulin, Maltose Binding Protein (MBP), Nus, Glutathione S Transferase (GST), Green Fluorescent Protein (GFP), Thioredoxin, S-tag, Softag 1 Softag 3, Strep, SBP (streptavidin binding peptide), Ty, V5, TC, and Glu-Glu, and combinations thereof.

16. The multiplex method of claim 1, wherein the first and second detection antibodies of step (e) each comprise a detectable label selected from the group consisting of horse radish peroxidase, quantum dots, fluorophores, alkaline phosphatase, and combinations thereof.

17. The multiplex method of claim 1, wherein the first and second detection antibodies of step (e) are first and second primary antibodies, wherein the first and second primary antibodies bind to (i) first and second secondary antibodies each comprising at least one detectable label or (ii) bind to first and second molecules each comprising at least one detectable label.

18. The multiplex method of claim 3, wherein the first and second detection antibodies of step (e) are phospho-tyrosine specific antibodies, and may be the same antibody.

19. The multiplex method of claim 4, wherein (i) the first detection antibody of step (e) is a phosphorylation-site specific antibody that specifically binds the first specific protein substrate when phosphorylated at a particular serine or threonine site but does not substantially bind the first specific protein substrate when not phosphorylated at that particular serine or threonine site, and wherein (ii) the second detection antibody of step (e) is a phosphorylation-site specific antibody that specifically binds the second specific protein substrate when phosphorylated at a particular serine or threonine site but does not substantially bind the second specific protein substrate when not phosphorylated at that particular serine or threonine site.

20. The multiplex method of claim 7, wherein the first detection antibody of step (e) is a phospho-tyrosine specific antibody, and wherein the second detection antibody of step (e) is a phosphorylation-site specific antibody that specifically binds the second specific protein substrate when phosphorylated at a particular serine or threonine site but does not substantially bind the second specific protein substrate when not phosphorylated at that particular serine or threonine site.

21. The multiplex method of claim 1, wherein the activities of the first and second target kinases that are assayed are substantially non-endogenous kinase activities.

22. The multiplex method of claim 1, wherein either or both of the first and second target kinases are mutant kinases or drug-resistant kinases.

23. A multiplex kit for assaying the effect of a test compound on the activity of multiple kinases in a biological cell, the kit comprising:
(a) a plurality of human embryonic kidney (HEK) 293 cells transiently transfected with one or more expression vectors comprising a plurality of polynucleotides encoding (i) a plurality of target kinases and (ii) a plurality of specific protein substrates of the target kinases, wherein each specific protein substrate is linked to at least one unique capture tag that is not conjugated to a rare earth element;
(b) one or more multi-well capture plates or beads comprising a plurality of capture antibodies, each of which specifically binds to one of the unique capture tags linked to a specific protein substrate;
(c) optionally, a plurality of human embryonic kidney (HEK) 293 cells transiently transfected with one or more expression vectors comprising a plurality of polynucleotides encoding (i) a plurality of target inactive mutant kinases and (ii) a plurality of specific protein substrates of the target kinases, wherein each specific protein substrate is linked to at least one unique capture tag that is not conjugated to a rare earth element, and wherein the expression vectors are suitable for transiently transfecting the cells;
(d) optionally, one or more reagents for culturing and/or lysing the cells transiently transfected with the expression vectors;
(e) optionally, a plurality of labeled detection antibodies each of which specifically binds to the phosphorylated form of a specific protein substrate but does not substantially bind to the un-phosphorylated form of that specific protein substrate;
(f) optionally, one or more control compounds for inhibiting one or more of the target kinases; and
(g) instructions for (i) culturing the transfected cells under conditions suitable to transiently express the plurality of target kinases and the plurality of specific protein substrates, (ii) plating the cells in multi-well plates, contacting the cells with at least one test compound, and incubating the cells under conditions suitable to allow phosphorylation of the specific protein substrates by the target kinases, (iii) lysing the cells, transferring the lysed cells to the multi-well capture plate of step (b), and capturing the plurality of specific protein substrates on or in the multi-well capture plate, (iv) detecting the presence of phosphorylated forms of the plurality of specific protein substrates using the plurality of labeled detection antibodies; and (v) comparing the level of phosphorylated forms of the plurality of specific protein substrates detected in a test sample of cells contacted with the test compound with 1) the level of phosphorylated forms of the plurality of specific protein substrates in at least one control sample taken from control HEK 293 cells not contacted with the test compound, and 2) the level of phosphorylated forms of the plurality of specific protein substrates in at least one control sample taken from control HEK 293 cells not contacted with the test compound but rather treated with known target kinase inhibitor(s), or otherwise equivalent cells expressing kinase-deficient mutant instead of active forms of the target kinases, thereby assaying the effect of the test compound on the activity of the plurality of target kinases within the cells contacted with the test compound.

24-28. (canceled)

29. A kit for assaying the effect of a test compound on the activity of one or more post-translational modification enzymes in a biological cell, the kit comprising:
(a) a plurality of human embryonic kidney (HEK) 293 cells transiently transfected with one or more expression vectors comprising a plurality of polynucleotides encoding (i) a plurality of target post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells;
(b) one or more multi-well capture plates or beads comprising a plurality of capture antibodies, each of which specifically binds to one of the unique capture tags linked to a specific protein substrate;
(c) optionally, a plurality of human embryonic kidney (HEK) 293 cells transiently transfected with one or more expression vectors comprising a plurality of polynucleotides encoding (i) a plurality of target inactive mutant post-translational modification enzymes and (ii) a plurality of specific protein substrates of the target post-translational modification enzymes, wherein each specific protein substrate is linked to at least one unique capture tag that is not conjugated to a rare earth element, and wherein the expression vectors are suitable for transiently transfecting the cells;
(d) optionally, one or more reagents for culturing and/or lysing the cells transiently transfected with the expression vectors;
(e) optionally, a plurality of labeled detection antibodies each of which specifically binds to the post-translationally modified form of a specific protein substrate but does not substantially bind to the unmodified form of that specific protein substrate;
(f) optionally, one or more control compounds for inhibiting the target post-translational modification enzymes; and
(g) instructions for (i) culturing the transfected cells under conditions suitable to transiently express the plurality of target post-translational modification enzymes and the plurality of specific protein substrates, (ii) plating the cells in multi-well plates, contacting the cells with at least one test compound, and incubating the cells under conditions suitable to allow post-translational modification of the specific protein substrates by the target post-translational modification enzymes, (iii) lysing the cells, transferring the lysed cells to the multi-well capture plate, and capturing the plurality of specific protein substrates on or in the multi-well capture plate, (iv) detecting the presence of post-translationally modified forms of the plurality of specific protein substrates using the plurality of labeled detection antibodies; and (v) comparing the level of post-translationally modified forms of the plurality of specific protein substrates detected in a test sample of cells contacted with the test compound with 1) the level of post-translationally modified forms of the plurality of specific protein substrates in at least one control sample taken from control HEK 293 cells not contacted with the test compound, and 2) the level of post-translationally modified forms of the plurality of specific protein substrates in at least one control sample taken from control HEK 293 cells not contacted with the test compound but rather treated with known target post-translational modification enzyme inhibitor(s), or otherwise equivalent cells expressing enzyme activity-deficient mutant instead of active forms of the target post-translational modification enzymes, thereby assaying the effect of the test compound on the activity of the plurality of post-translational modification enzymes within the cells contacted with the test compound.

30-37. (canceled)

38. A method for assaying the effect of a test compound on the activity of a post-translational modification enzyme in a biological cell, the method comprising the steps of:
(a) obtaining a plurality of transiently transfected biological cells that transiently express (i) at least one target post-translational modification enzyme and (ii) at least one specific protein substrate of the target post-translational modification enzyme, wherein the specific protein substrate is linked to at least one capture tag that is not conjugated to a rare earth element;
(b) culturing the plurality of biological cells under conditions suitable to transiently express the target post-translational modification enzyme and the specific protein substrate;
(c) incubating the plurality of biological cells with at least one test compound under conditions suitable to allow post-translational modification of the specific protein substrate by the target post-translational modification enzyme;
(d) lysing the plurality of biological cells and capturing the specific protein substrate using at least one capture antibody that specifically binds the capture tag;
(e) detecting the presence of post-translationally modified specific protein substrate using at least one detection antibody that specifically binds the post-translationally-modified form of the specific protein substrate but does not substantially bind the specific protein substrate when not post-translationally modified; and
(f) comparing the level of post-translationally modified specific protein substrate detected in step (e) with 1) the level of post-translationally modified specific protein substrate in at least one control sample taken from control biological cells not contacted with the test compound, and 2) the level of post-translationally modified specific protein substrate in at least one control sample taken from control biological cells not contacted with the test compound but rather treated with known target post-translational modification enzyme inhibitor(s), or otherwise equivalent cells expressing enzyme activity-deficient mutant instead of active forms of the target post-translational modification enzyme(s), thereby assaying the effect of the test compound on the activity of the target post-translational modification enzyme within the biological cells of step (c).

39-79. (canceled)
